(19) United States
(12) Patent Application Publication
(10) Pub. No.: US 2007/0258907 A1
(43) Pub. Date: Nov. 8, 2007

(54) Polymer-coated paramagnetic particles

(76) Inventor: Mark E. Davis, Pasadena, CA (US)

Correspondence Address: ROPES & GRAY LLP;PATENT DOCKETING 39/41, ONE INTERNATIONAL PLACE, BOSTON, MA, 02110-2624, US

(21) Appl. No.: 11/789,470

(22) Filed: Apr. 24, 2007

Related U.S. Application Data
(60) Provisional application No. 60/794,792, filed on Apr. 24, 2006.

(51) Int. Cl. A61K 49/10

(52) U.S. Cl. 424/9.322; 534/15

(57) ABSTRACT

The present invention relates to novel compositions of polymer-coated paramagnetic particles, defined as paramagnetic particles non-covalently coated with polymeric materials, which optionally possess targeting ligands, therapeutic agents, or carrier ligands. By selecting from a variety of linker groups and targeting ligands the coated paramagnetic particles are suitable for a wide variety of methods for controlled delivery of the particles. The invention also relates to methods and uses of imaging, diagnosing, and treating diseased or cancerous tissue using the particles.

### CROSS-REFERENCE TO RELATED APPLICATIONS

This patent application claims the benefit of U.S. Provisional Application No. 60/794,792 filed on Apr. 24, 2006. The specification of this application is incorporated herein by reference in its entirety.

### STATEMENT REGARDING FEDERALLY SPONSORED RESEARCH OR DEVELOPMENT

The U.S. Government has certain rights in this invention pursuant to Grant No. EB004657 awarded by the National Institutes of Health.

### BACKGROUND OF THE INVENTION

In magnetic resonance imaging (MRI) an image of an organ or tissue is obtained by placing a subject in a strong magnetic field and observing the interactions between the magnetic spins of the protons and radiofrequency electromagnetic radiation. (For a review of MR imaging technique see Balter, S. RadioGraphics 1987, 7 (2) 371-383; Fullerton, G. D. RadioGraphics 1987, 7 (3), 579-596). Two parameters termed proton relaxation times are of primary importance in the generation of the image. They are called T₁ (also called the spin-lattice or longitudinal relaxation time) and T₂ (the spin-spin or transverse relaxation time). T₁ and T₂ depend on the chemical and physical environment of protons in various organs or tissues.

The utility of MR imaging techniques in the characterization and differentiation of pathologic from healthy tissues is most easily demonstrated in cases where divergent relaxation times occur within a region of interest. For example in cerebral tissue the protons of the cerebral spinal fluid have far different relaxation times from neural tissue and the resulting MR images are of high contrast.

In other instances the image produced may lack definition and clarity due to a similarity of the signal from different tissues or different compartments within a tissue. In some cases, the magnitude of these differences is small, limiting the diagnostic effectiveness of MR imaging. Thus, there exists a real need for methods which increase or magnify these differences. One approach to improving image quality is through the use of contrast agents.

Contrast agents are substances which exert an effect on the NMR parameters of various chemical species around them. Ordinarily, these effects are strongest on the species closest to the agent, and decrease as the distance from the agent is increased. Thus, the areas closest to the agent will possess NMR parameters which are different from those further away. Proper choice of a contrast agent will, theoretically, result in uptake by only a certain portion of the organ or a certain type of tissue (e.g., diseased tissues), thus providing an enhancement of the contrast, which in turn generates a more accurate image.

Since NMR images can be generated from an analysis of the T₁ or T₂ parameters discussed above, it is desirable to have a contrast agent which affects either or both parameters. Much research has, therefore, centered around two general classes of magnetically active materials: paramagnetic materials (which act primarily to decrease T₁) and ferromagnetic materials (which act primarily to decrease T₂).

Paramagnetism occurs in materials that contain unpaired electrons which do not interact and are not coupled. Paramagnetic materials are characterized by a weak magnetic susceptibility, where susceptibility is the degree of response to an applied magnetic field. They become weakly magnetic in the presence of a magnetic field, and rapidly lose such activity (i.e., demagnetize) once the external field is removed. It has long been recognized that the addition of paramagnetic solutes to water causes a decrease in the T₁ parameter.

Because of such effects on T₁ a number of paramagnetic materials have been used as NMR contrast agents. However, a major problem with the use of contrast agents for imaging is that many of the paramagnetic and ferromagnetic materials exert toxic effects on biological systems making them inappropriate for in vivo use. Because of problems inherent with the use of many presently available contrast agents, there exists a need for new agents adaptable for clinical use. In order to be suitable for in vivo diagnostic use, such agents must combine low toxicity with an array of properties including superior contrasting ability, ease of administration, specific biodistribution (permitting a variety of organs to be targeted), and a size sufficiently small to permit free circulation through a subject's vascular system (a typical route for delivery of the agent to various organs). Additionally, the agents must be stable in vivo for a sufficient time to permit the clinical study to be accomplished, yet capable of being ultimately metabolized and/or excreted by the subject.

### SUMMARY OF THE INVENTION

The present invention relates to novel compositions of polymer-coated paramagnetic particles, defined as paramagnetic particles non-covalently coated with polymeric materials, which optionally possess targeting ligands, therapeutic agents, or carrier ligands, and to diagnostic and therapeutic uses of such particles. In certain embodiments, the particles are covalently and/or non-covalently coupled to targeting ligands. In certain embodiments, the particles are covalently and/or non-covalently coupled to therapeutic agents, as carriers for therapeutics delivery.

In one aspect, the present invention provides water-soluble, biocompatible polymer-coated paramagnetic particles covalently attached to targeting ligands that target the particles to a particular organ or tissue of interest.

In another aspect, the present invention provides water-soluble, biocompatible polymer-coated paramagnetic particles covalently attached to bioactive moieties through attachments that are cleaved under biological conditions to release the bioactive moieties. In certain such embodiments, the polymer comprises cyclic host moieties alternating with linker moieties that connect the cyclic host structures, e.g., into linear or branched polymers, preferably linear polymers. The polymer may be a polycation, polyanion, or non-ionic polymer. The bioactive agent, which may be a therapeutic agent, a diagnostic agent, or an adjuvant, preferably makes up at least 5%, 10%, 15%, 20%, 25%, 30%, or even at least 35% by weight of the conjugate. In certain embodiments, the rate of drug release is dependent primarily upon the rate of hydrolysis. In certain other embodiments, the rate of drug release is dependent primarily on enzymatic cleavage.

The present invention provides a paramagnetic particle coated by a cyclodextrin-containing polymer for use in monitoring the in vivo biodistribution and pharmacokinetics of novel therapeutic agents non-invasively by MR imaging.

The present invention also provides a paramagnetic particle coated by a cyclodextrin-containing polymer for use in drug delivery of these therapeutic agents. The invention also provides compounds for use in controlled drug delivery which are capable of releasing a therapeutic agent in a targeted, predictable, and controlled rate.

Accordingly, one aspect of the present invention is a paramagnetic particle coated by a polymer comprising cyclodextrin moieties, a therapeutic agent, and an optional ligand-targeting agent. The polymer may be linear or branched, and may be formed via polycondensation of cyclodextrin-containing monomers, or by copolymerization between one or more cyclodextrin-containing monomers and one or more comonomers which do not contain cyclodextrin moieties. Furthermore, the present invention also contemplates the use of cyclodextrin-containing polymers formed by grafting cyclodextrin moieties to a polymer. The cyclodextrin moieties contemplated by the present invention include, but are not limited to, α, β, and γ cyclodextrins and oxidized forms thereof. Depending on the drug/polymer ratio desired, the therapeutic agent may be covalently attached to a monomer via an optional linker prior to the polymerization step, or may be subsequently grafted onto the polymer via an optional linker, or may be non-covalently attached to the polymer as an inclusion complex or other host-guest interaction. Likewise, the targeting ligand may be covalently attached to a monomer via an optional linker prior the polymerization step, or may be subsequently grafted onto the polymer via an optional linker, or may be non-covalently attached to the polymer as an inclusion complex or other host-guest interaction.

In another aspect, the particles are covalently coupled to carrier ligands which optionally chelate one or more radioisotopes. Such embodiments are useful for dual imaging with MRI and positron emission tomography (PET). Dual imaging can be useful for the diagnosis of diseased or cancerous tissue.

In another aspect, the particle may be modified by one or more inclusion guests comprising a first portion that forms an inclusion complex with the cyclodextrin moieties of the polymer, and a second portion that comprises a surface-modifying group. In certain aspects, the surface modifying group further comprises a targeting ligand or a therapeutic agent. In certain aspects, the surface modifying group comprises a biohydrolyzable bond that is cleaved under biological conditions to release the therapeutic agent.

To illustrate further, one embodiment of the invention is a paramagnetic particle coated by a cyclodextrin-containing polymeric compound wherein the cyclodextrin-containing polymer comprises n′ units of U, wherein n′ represents an integer in the range of 1 to about 30,000; and U is represented by the general formula: 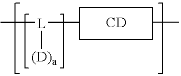
wherein,

CD represents a cyclodextrin molecule, or derivative thereof;

L represents a linker group;

D, independently for each occurrence, represents a targeting ligand, a therapeutic agent or prodrug thereof, or a carrier ligand;

a, independently for each occurrence, represents an integer in the range of 0 and 10 (preferably 1 to 8, 1 to 5, or even 1 to 3).

In certain embodiments, a surface of the polymer is modified by one or more inclusion guests, each inclusion guest comprising a first portion that forms an inclusion complex with the cyclodextrin moieties of the polymer, and a second portion that comprises a surface-modifying group.

In certain embodiments, the paramagnetic particle is a superparamagnetic particle.

In certain embodiments, the cyclodextrin-containing polymer coats a plurality of paramagnetic particles, e.g., in a single superparamagnetic particle.

In certain embodiments, the paramagnetic particle comprises a metal oxide or metal mixed oxide. In certain embodiments, the metal oxide is a mixed oxide. In certain embodiments, the metals of the mixed oxide are selected from aluminum, barium, beryllium, chromium, cobalt, copper, iron, manganese, magnesium, strontium, zinc, rare earth metals, trivalent metal ions or mixtures thereof. In certain preferred embodiments, the metal of the paramagnetic particle is iron.

In certain embodiments, the particle has an average hydrodynamic diameter in the range of 10-100 nanometers. In certain preferred embodiments the particle has an average hydrodynamic diameter of about 30 nanometers (e.g., 20 to 40 nm). In certain preferred embodiments, the particle has an average hydrodynamic diameter of about 90 nanometers (e.g., 80 to 100 nm).

In certain embodiments, the linker group or linker moiety represents an alkylene group wherein one or more methylene groups is optionally replaced by a group Y, wherein each Y, independently for each occurrence, is selected from substituted or unsubstituted aryl, heteroaryl, cycloalkyl, heterocycloalkyl, or —O—, C(═X) (wherein X is NR₁, O or S), —OC(O)—, —C(═O)O, —NR₁—, —NR₁CO—, —C(O)NR₁—, —S(O)ₙ— (wherein n is 0, 1, or 2), —OC(O)—NR₁, —NR₁—C(O)—NR₁—, —NR₁—C(NR₁)—NR₁—, and —B(OR₁)— (wherein R₁, independently for each occurrence, represents H or a lower alkyl), selected such that no two heteroatoms are adjacent to each other.

In certain embodiments, the linker group or linker moiety comprises an amino acid or peptide.

In certain embodiments, a plurality of occurrences of D in general formula U independently represent a targeting ligand. In certain embodiments, the targeting ligand is transferrin. In certain embodiments, a plurality of occurrences of D in general formula U independently represent a therapeutic agent.

In certain embodiments, the paramagnetic particle is targeted to a tumor.

In certain embodiments, a plurality of occurrences of D in general formula U independently represent a therapeutic agent. In certain embodiments, the therapeutic agent is selected from an anti-cancer, anti-fungal, anti-bacterial, anti-mycotic, or anti-viral therapeutic.

In certain embodiments, D in general formula U. independently for each occurrence, is a receptor agonist.

In certain embodiments, D in general formula U, independently for each occurrence, is a receptor antagonist.

In certain embodiments, the therapeutic agent is a small molecule, a peptide, a protein, or a polymer.

In certain embodiments, D in general formula U is attached to the cyclodextrin polymer by a linker that comprises a biohydrolyzable bond.

In certain embodiments, the biohydrolyzable bond is selected from an ester, amide, carbonate, or a carbamate.

In certain embodiments, D in general formula U, independently for each occurrence, is a carrier ligand.

In certain embodiments, the carrier ligand is a radioisotope carrier, e.g., for contrast imaging.

In certain embodiments, the radioisotope carrier is selected from chelators, capsules or combinations thereof.

In certain embodiments, the chelators are selected from 1,4,7,10-tetraazacyclododecane-N,N′,N″,N′″-tetraacetic acid (DOTA), tetra-butyl-calix[4]arene-crown-6-dicarboxylic acid (TBBCDA), 5,11,17,23-tetra-t-butyl-25,26,27,28-tetrakis (carboxymethoxy)-calix[6]arene (HBHC), diethylenetriamine-pentaacetic acid (DTPA), EDTA, or combinations thereof.

In certain embodiments, the radioisotope carrier chelates a radioisotope selected from yttrium-90, indium-111, radium-23, actinium-225, bismuth-212, bismuth-213, scandium-47, astatine-211, rhenium-186, rhenium-188, iodine-131, iodine-124, lutetium-177, holmium-166, samarium-153, copper-64, copper-67, phosphorus-32 or combinations thereof.

In certain embodiments, the carrier ligand further comprises a contrast agent.

In certain embodiments, the contrast agent comprises a gamma-emitting radioisotope. In certain embodiments, the gamma-emitting radioisotope is selected from arsenic-74, copper-64, copper-67, fluorine-18, gallium-67, indium-111, iodine-131, rhenium-186, rhenium-188, technetium-99m, thorium-201, yttrium-86, yttrium-91, zirconium-89 or combinations thereof.

In certain embodiments, the polymer comprises a gamma-emitting radioisotope. In certain embodiments, the gamma-emitting radioisotope is selected from arsenic-74, copper-64, copper-67, fluorine-18, gallium-67, indium-111, iodine-131, rhenium-186, rhenium-188, technetium-99m, thorium-201, yttrium-86, yttrium-91, zirconium-89 or combinations thereof.

In certain embodiments, the inclusion guest is adamantane.

In certain embodiments, the inclusion guest is covalently attached to a surface-modifying group.

In certain embodiments, the surface-modifying group further comprises a ligand.

In certain embodiments, the surface-modifying group comprises a biohydrolyzable bond.

In certain embodiments, the ligand is a targeting ligand. In certain embodiments, the targeting ligand is transferrin.

In certain embodiments, the ligand is a therapeutic agent or prodrug thereof.

In certain embodiments, the therapeutic agent is selected from an anti-cancer, anti-fungal, anti-bacterial, anti-mycotic, or anti-viral therapeutic. In certain embodiments, the therapeutic agent is a receptor agonist or a receptor antagonist. In certain embodiments, the therapeutic agent is a small molecule, a peptide, a protein, or a polymer.

In certain embodiments, the surface-modifying group is a stabilizing group. In certain embodiments, the stabilizing group is selected from phosphate, diphosphate, carboxylate, polyphosphate, thiophosphate, phosphonate, thiophosphonate, sulphate, sulphonate, mercapto, silanetriol, trialkoxysilane-containing polyalkylene glycols, carbohydrate or phosphate-containing nucleotides, oligomers thereof or polymers thereof. In certain preferred embodiments, the stabilizing group is polyethylene glycol.

Another aspect of the present invention is a method for preparing the paramagnetic particle coated by cyclodextrin-containing polymeric conjugates described herein.

Another aspect of the present invention is a pharmaceutical composition comprising a polymer-coated paramagnetic particle as discussed above.

Another aspect of the present invention is a pharmaceutical dosage form comprising a polymer-coated paramagnetic particle as described herein.

Another aspect of the present invention is a method for magnetic resonance imaging using the polymer-coated paramagnetic particle as described herein.

Another aspect of the present invention is a method for dual magnetic resonance imaging/positron emission tomography using the polymer-coated paramagnetic particle as described herein.

Another aspect of the present invention is a method for treating a subject comprising administering a therapeutically effective amount of any of the polymer-coated paramagnetic particles described herein.

Another aspect of the present invention is a method for the use of a one or more of the polymer-coated paramagnetic particles for diagnosing a disease in a patient.

### BRIEF DESCRIPTION OF THE FIGURES

FIG. 1 schematically depicts the combination of an unmodified polymer with an iron oxide paramagnetic particle to yield a preformed polyplex, in which the particle is non-covalently coated with the polymer.

FIG. 2 illustrates the addition of adamantane modified with a polyethylene glycol (PEG) group (PEG-Ad) with the preformed polyplex to yield a stabilized polyplex, in which the cyclodextrin moieties of the polymer that coats the paramagnetic particle form host-guest interactions with the adamantane moities.

FIG. 3 depicts the addition of adamantane modified with PEG further derivatized with a ligand (Ligand-PEG-Ad) to the preformed polyplex.

FIG. 4 illustrates the combination of both PEG-Ad and Ligand-PEG-Ad to the preformed polyplex to form a mixed stabilized polyplex.

FIG. 5 schematically depicts the preparation of a mixed modified stabilized polyplex labeled with copper radioisotope. Step A shows the covalent modification of a polymer with DOTA comprising a linker moiety. Step B depicts the coating of an iron oxide particle with the DOTA-modified polymer from A to yield a DOTA-modified preformed polyplex. Step C depicts the chelation of a radioisotope of copper (Cu) to the DOTA moiety of the DOTA-modified preformed polyplex. Step D shows the addition of both PEG-Ad and Ligand-PEG-Ad to the copper-loaded DOTA-modified preformed polyplex, resulting in a Cu-loaded DOTA-modified mixed stabilized polyplex.

FIG. 6 shows the hydrodynamic diameter of the nanoparticles as measured by dynamic light scattering (DLS). Nanoparticles diluted into water have a larger hydrodynamic radius than nanoparticles diluted into PBS. Pegylation increases the hydrodynamic diameter of the nanoparticles.

FIGS. 7A and B are histograms showing the nanoparticle hydrodynamic diameter distributions. FIG. 7A shows the smaller F1 particles have a hydrodynamic diameter of around 31 nm. FIG. 7B shows the F3 particles have a hydrodynamic diameter of around 95 nm.

FIG. 8 shows the zeta potential of the nanoparticles in water. Pegylation reduces the negative charge of the nanoparticles.

FIGS. 9A and B show TEM images of CD-polymer coated iron oxide nanoparticles. FIG. 9A shows a TEM image of F1 particles. Iron oxide cores are roughly 5 nm in diameter. FIG. 9B shows an image of F3 particles. Iron oxide cores are roughly 10 nm in diameter.

FIG. 10 shows T₂-weighted MR image of CD-coated iron oxide nanoparticles with a TE (echo time) of 21 msec. Nanoparticles cause a concentration-dependent darkening effect on the MR images. The top number indicates the concentration of iron in μg Fe/mL.

FIG. 11 shows a plot of iron concentration versus inverse T₂ relaxation time.

FIG. 12 shows a plot of iron concentration versus 1/T₁.

FIG. 13 shows the in vitro uptake of imaging agents into RAW264.7 mouse macrophage cells. Pegylation reduces macrophage uptake. The smaller F1 particles are taken up by macrophages to a greater extent than the larger F3 particles.

FIG. 14 shows a summary of MR data for F3 contrast agents injected into A/J mice bearing Neuro2A subcutaneous tumor. Post-injection scans were taken roughly 1 h after contrast agent injection. Variables are dose of injected nanoparticles, targeting ligand, and time after injection.

FIGS. 15A and B show T₂-weighted MR image of subcutaneous Neuro 2A tumor on the back of an A/J mouse. FIG. 15A shows a pre-contrast agent image. FIG. 15B shows an image taken 2 h after systemic injection of F3-AD-PEG-Tf (1.7 w % Tf) (12.5 mg Fe/kg tissue).

FIG. 16A-D show T₂-weighted MR images of mouse livers. FIGS. 16A and B were taken prior to injection of contrast agent. FIG. 16C shows the liver 4 h after systemic injection of F1 contrast agent (1.6 mg Fe/kg tissue). FIG. 16D shows the liver 4 h after systemic injection of Feridex (1.6 mg Fe/kg tissue). Both contrast agents preferentially localize to the liver and cause a similar drop in the tissue contrast (˜90% drop in contrast for F1 versus ˜50% drop in contrast for Feridex).

FIG. 17 shows HE staining of a Hep3B tumor cross-section. Small, black cells are macrophages, and larger pink cells are tumors. Macrophages are localized to the necrotic ‘groove’ in the tumor, which corresponds to the darkened region of the tumor by MRI shown in FIG. 18.

FIGS. 18A and B shows a T₂-weighted MR image of subcutaneous Hep3B tumor on the back of a nude mouse. FIG. 18A shows an image of a pre-contrast agent image.

FIG. 18B shows an image taken 2 h after systemic injection of F3-AD-PEG-Tf (1.7 w % Tf) (12.5 mg Fe/kg tissue). Notice the darkening of the necrotic region of the tumor (arrow) after injection of contrast agent.

FIG. 19 shows HE staining of a Neuro2A tumor cross-section. Small, black cells are macrophages, and larger pink cells are tumors. Macrophages appear throughout the tumor.

FIG. 20 shows the distribution of F3-AD-PEG particles following injection into A/J mice with Neuro2A tumors expressed as mean percent injected dose per gram. The distribution is detected by PET scanning of the ⁶⁴Cu label.

FIG. 21 shows the distribution of F3-AD-PEG-Tf particles following injection into A/J mice with Neuro2A tumors expressed as mean percent injected dose per gram. The distribution is detected by PET scanning of the ⁶⁴Cu label.

### DETAILED DESCRIPTION OF THE INVENTION

I. Overview

The present invention relates to novel compositions of paramagnetic particles coated with cyclodextrin-containing polymer as contrast agents for MR imaging. In certain embodiments, the particles are modified with targeting ligands that target the particles to particular organs or tissues. In certain embodiments, the particles are designed for drug delivery of therapeutic agents. In certain embodiments, the paramagnetic particles are modified by inclusion guests, wherein the inclusion guests form inclusion complexes with cyclodextrin moieties of the polymer and comprise a surface-modifying group. In certain embodiments, the surface modifying group(s) reduce macrophage uptake of the paramagnetic particles.

In certain embodiments, a polymer-coated paramagnetic particle may comprise multiple paramagnetic particles, e.g., distinct particles of paramagnetic material, within the polymer coating. The multiple paramagnetic particles may comprise one type or a plurality of types of paramagnetic material, e.g., different paramagnetic particles in a polymer-coated paramagnetic particle may comprise different metal oxides and/or mixed metal oxides, e.g., such that a single polymer-coated paramagnetic particle comprises two or more paramagnetic particles of different chemical composition. In certain embodiments, the multiple paramagnetic particles may be in intimate contact with each other, or even compressed into a compound solid. In certain embodiments, the multiple paramagnetic particles may be separated by a layer of polymer coating or other adhesive or filler material.

In certain embodiments, these cyclodextrin-containing polymers improve drug stability and/or solubility, and/or reduce toxicity, and/or improve efficacy of the small molecule therapeutic when used in vivo. In certain embodiments, the polymers can be used for delivery of therapeutics such as camptothecin, taxol, doxorubicin, and amphotericin. Furthermore, by selecting from a variety of linker groups, and/or targeting ligands, the rate of drug release from the polymers can be attenuated for controlled delivery. The invention also relates to methods of treating subjects with the therapeutic compositions described herein.

More generally, the present invention provides water-soluble, biocompatible polymer-coated paramagnetic particles comprising a paramagnetic particle coated by a water-soluble, biocompatible cyclodextrin-containing polymer. These particles are optionally modified covalently or non-covalently with therapeutic agents through linker moieties that are cleaved under biological conditions to release the bioactive moieties. In certain embodiments, the particles are also modified with radioactive agents for use in PET scanning.

The bioactive agent, which may be a therapeutic agent, a diagnostic agent, or an adjuvant (such as a radiosensitizer, or a compound that lacks significant activity administered alone but that potentiates the activity of another therapeutic agent), preferably makes up at least 5%, 10%, 15%, 20%, 25%, 30%, or even 35% by weight of the conjugate. In preferred embodiments, administration of the polymer to a patient results in release of the bioactive agent over a period of at least 6 hours, preferably at least 12 or 18 hours. For example, the agent may be released over a period of time ranging from 6 hours to a month, 6 hours to two weeks, 6 hours to 3 days, etc. In certain embodiments, the rate of drug release is dependent primarily upon the rate of hydrolysis (as opposed to enzymatic cleavage), e.g., the rate of release changes by less than a factor of 5, preferably less than a factor of 2, in the presence of hydrolytic enzymes. In other embodiments, the rate of drug release may be dependent primarily on the rate of enzymatic cleavage.

Paramagnetic particles coated by cyclodextrin-containing polymeric conjugates of the present invention may be useful to improve solubility and/or stability of a bioactive/therapeutic agent, reduce drug-drug interactions, reduce interactions with blood elements including plasma proteins, reduce or eliminate immunogenicity, protect the agent from metabolism, modulate drug-release kinetics, improve circulation time, improve drug half-life (e.g., in the serum, or in selected tissues, such as tumors), attenuate toxicity, improve efficacy, normalize drug metabolism across subjects of different species, ethnicities, and/or races, and/or provide for targeted delivery into specific cells or tissues. Poorly soluble and/or toxic compounds may benefit particularly from incorporation into polymeric particles of the invention.

II. Definitions

(a) General Terms

An ‘adjuvant’, as the term is used herein, is a compound that has little or no therapeutic value on its own, but increases the effectiveness of a therapeutic agent. Exemplary adjuvants include radiosensitizers, transfection-enhancing agents (such as chloroquine and analogs thereof), chemotacetic agents and chemoattractants, peptides that modulate cell adhesion and/or cell mobility, cell permeabilizing agents, inhibitors of multidrug resistance and/or efflux pumps, etc.

The term “agonist”, as used herein, is meant to refer to an agent that mimics or up-regulates (e.g., potentiates or supplements) the bioactivity of a protein of interest, or an agent that facilitates or promotes (e.g., potentiates or supplements) an interaction among polypeptides or between a polypeptide and another molecule (e.g., a steroid, hormone, nucleic acids, small molecules etc.). An agonist can be a wild-type protein or derivative thereof having at least one bioactivity of the wild-type protein. An agonist can also be a small molecule that up-regulates the expression of a gene or which increases at least one bioactivity of a protein. An agonist can also be a protein or small molecule which increases the interaction of a polypeptide of interest with another molecule, e.g., a target peptide or nucleic acid.

“Antagonist” as used herein is meant to refer to an agent that down-regulates (e.g., suppresses or inhibits) the bioactivity of a protein of interest, or an agent that inhibits/suppresses or reduces (e.g., destabilizes or decreases) interaction among polypeptides or other molecules (e.g., steroids, hormones, nucleic acids, etc.). An antagonist can also be a compound that down-regulates the expression of a gene of interest or which reduces the amount of the wild-type protein present. An antagonist can also be a protein or small molecule which decreases or inhibits the interaction of a polypeptide of interest with another molecule, e.g., a target peptide or nucleic acid.

The terms “biocompatible polymer” and “biocompatibility” when used in relation to polymers are art-recognized. For example, biocompatible polymers include polymers that are neither themselves toxic to the host (e.g., an animal or human), nor degrade (if the polymer degrades) at a rate that produces monomeric or oligomeric subunits or other byproducts at toxic concentrations in the host. In certain embodiments of the present invention, biodegradation generally involves degradation of the polymer in an organism, e.g., into its monomeric subunits, which may be known to be effectively non-toxic. Intermediate oligomeric products resulting from such degradation may have different toxicological properties, however, or biodegradation may involve oxidation or other biochemical reactions that generate molecules other than monomeric subunits of the polymer. Consequently, in certain embodiments, toxicology of a biodegradable polymer intended for in vivo use, such as implantation or injection into a patient, may be determined after one or more toxicity analyses. It is not necessary that any subject composition have a purity of 100% to be deemed biocompatible. Hence, a subject composition may comprise 99%, 98%, 97%, 96%, 95%, 90% 85%, 80%, 75% or even less of biocompatible polymers, e.g., including polymers and other materials and excipients described herein, and still be biocompatible.

To determine whether a polymer or other material is biocompatible, it may be necessary to conduct a toxicity analysis. Such assays are well known in the art. One example of such an assay may be performed with live carcinoma cells, such as GT3TKB tumor cells, in the following manner: the sample is degraded in 1 M NaOH at 37° C. until complete degradation is observed. The solution is then neutralized with 1 M HCl. About 200 μL of various concentrations of the degraded sample products are placed in 96-well tissue culture plates and seeded with human gastric carcinoma cells (GT3TKB) at 104/well density. The degraded sample products are incubated with the GT3TKB cells for 48 hours. The results of the assay may be plotted as % relative growth vs. concentration of degraded sample in the tissue-culture well. In addition, polymers and formulations of the present invention may also be evaluated by well-known in vivo tests, such as subcutaneous implantations in rats to confirm that they do not cause significant levels of irritation or inflammation at the subcutaneous implantation sites.

The term “biodegradable” is art-recognized, and includes polymers, compositions and formulations, such as those described herein, that are intended to degrade during use. Biodegradable polymers typically differ from non-biodegradable polymers in that the former may be degraded during use. In certain embodiments, such use involves in vivo use, such as in vivo therapy, and in other certain embodiments, such use involves in vitro use. In general, degradation attributable to biodegradability involves the degradation of a biodegradable polymer into its component subunits, or digestion, e.g., by a biochemical process, of the polymer into smaller, non-polymeric subunits. In certain embodiments, two different types of biodegradation may generally be identified. For example, one type of biodegradation may involve cleavage of bonds (whether covalent or otherwise) in the polymer backbone. In such biodegradation, monomers and oligomers typically result, and even more typically, such biodegradation occurs by cleavage of a bond connecting one or more of subunits of a polymer. In contrast, another type of biodegradation may involve cleavage of a bond (whether covalent or otherwise) internal to sidechain or that connects a side chain to the polymer backbone. For example, a therapeutic agent or other chemical moiety attached as a side chain to the polymer backbone may be released by biodegradation. In certain embodiments, one or the other or both general types of biodegradation may occur during use of a polymer.

As used herein, the term “biodegradation” encompasses both general types of biodegradation. The degradation rate of a biodegradable polymer often depends in part on a variety of factors, including the chemical identity of the linkage responsible for any degradation, the molecular weight, crystallinity, biostability, and degree of cross-linking of such polymer, the physical characteristics (e.g., shape and size) of an implant, and the mode and location of administration. For example, the greater the molecular weight, the higher the degree of crystallinity, and/or the greater the biostability, the biodegradation of any biodegradable polymer is usually slower. The term “biodegradable” is intended to cover materials and processes also termed “bioerodible”.

In certain embodiments wherein the biodegradable polymer also has a therapeutic agent or other material associated with it, the biodegradation rate of such polymer may be characterized by a release rate of such materials. In such circumstances, the biodegradation rate may depend on not only the chemical identity and physical characteristics of the polymer, but also on the identity of material(s) incorporated therein. Degradation of the subject compositions includes not only the cleavage of intramolecular bonds, e.g., by oxidation and/or hydrolysis, but also the disruption of intermolecular bonds, such as dissociation of host/guest complexes by competitive complex formation with foreign inclusion hosts.

In certain embodiments, polymeric formulations of the present invention biodegrade within a period that is acceptable in the desired application. In certain embodiments, such as in vivo therapy, such degradation occurs in a period usually less than about five years, one year, six months, three months, one month, fifteen days, five days, three days, or even one day on exposure to a physiological solution with a pH between 6 and 8 having a temperature of between 25 and 37° C. In other embodiments, the polymer degrades in a period of between about one hour and several weeks, depending on the desired application.

As used herein the term “bioerodable” refers to polymers which deliver sustained effective amounts of therapeutic agent to target tissue over desired extended periods of time. Thus, a polymer according to the invention in the biological environment of host tissue and the like, in one aspect, is subjected to hydrolytic enzymes and oxidative species under, and in proportion to, the host's inflammatory response. This results in release of the therapeutic agent via the breaking of the covalent linked bonds. Thus, in certain embodiments, the materials of the invention utilize the mammal's own wound-healing repair process in being degraded thereby, as hereinbefore described.

The biodegradable polymers polylactic acid, polyglycolic acid, and polylactic-glycolic acid copolymer (PLGA), have been investigated extensively for nanoparticle formulation. These polymers are polyesters that, upon implantation in the body, undergo simple hydrolysis. The products of such hydrolysis are biologically compatible and metabolizable moieties (e.g., lactic acid and glycolic acid), which are eventually removed from the body by the citric acid cycle. Polymer biodegradation products are formed at a very slow rate, and hence do not affect normal cell function. Several implant studies with these polymers have proven safe in drug delivery applications, used in the form of matrices, microspheres, bone implant materials, surgical sutures, and also in contraceptive applications for long-term effects. These polymers are also used as graft materials for artificial organs, and recently as basement membranes in tissue engineering investigations. Nature Med. 824-826 (1996). Thus, these polymers have been time-tested in various applications and proven safe for human use. Most importantly, these polymers are FDA-approved for human use.

When polymers are used for delivery of pharmacologically active agents in vivo, it is essential that the polymers themselves be nontoxic and that they degrade into non-toxic degradation products as the polymer is eroded by the body fluids. Many synthetic biodegradable polymers, however, yield oligomers and monomers upon erosion in vivo that adversely interact with the surrounding tissue. D. F. Williams, J. Mater. Sci. 1233 (1982). To minimize the toxicity of the intact polymer carrier and its degradation products, polymers have been designed based on naturally occurring metabolites. Probably the most extensively studied examples of such polymers are the polyesters derived from lactic or glycolic acid and polyamides derived from amino acids.

A number of bioerodable or biodegradable polymers are known and used for controlled release of pharmaceuticals. Such polymers are described in, for example, U.S. Pat. Nos. 4,291,013, 4,347,234, 4,525,495, 4,570,629, 4,572,832, 4,587,268, 4,638,045, 4,675,381, 4,745,160, 5,219,980.

A biohydrolyzable bond (e.g., ester, amide, carbonate, carbamates, or imide) refers to a bond that is cleaved (e.g., an ester is cleaved to form a hydroxyl and a carboxylic acid) under physiological conditions. Physiological conditions include the acidic and basic environments of the digestive tract (e.g., stomach, intestines, etc.), acidic environment of a tumor, enzymatic cleavage, metabolism, and other biological processes, and preferably refer to physiological conditions in a vertebrate, such as a mammal.

As used herein the terms “comonomer A”, “linker”, “linker group”, and “linker moiety” refer to any straight chain or branched, symmetric or asymmetric compound which upon reaction with a cyclodextrin monomer precursor or other suitable cyclic moiety links two such moieties together. In certain embodiments, the linker group is a compound containing at least two functional groups through which reaction and thus linkage of the cyclodextrin monomers can be achieved. Examples of functional groups, which may be the same or different, terminal or internal, of each linker group include, but are not limited, to amino, acid, imidazole, hydroxyl, thio, acyl halide, —C═C—, or —C≡C— groups and derivatives thereof. In preferred embodiments, the two functional groups are the same and are located at termini of the comonomer. In certain embodiments, a linker group contains one or more pendant groups with at least one functional group through which reaction and thus linkage of therapeutic agent or targeting ligand can be achieved, or branched polymerization can be achieved. Examples of functional groups, which may be the same or different, terminal or internal, of each linker group pendant group include, but are not limited, to amino, acid, imidazole, hydroxyl, thiol, acyl halide, ethylene, and ethyne groups and derivatives thereof. In certain embodiments, the pendant group is a (un)substituted branched, cyclic or straight chain C1-C10 (preferably C1-C6) alkyl, or arylalkyl optionally containing one or more heteroatoms, e.g., N, O, S, within the chain or ring.

Upon copolymerization of a linker group with a cyclodextrin monomer precursor, two cyclodextrin monomers may be linked together by joining the primary hydroxyl side of one cyclodextrin monomer with the primary hydroxyl side of another cyclodextrin monomer, by joining the secondary hydroxyl side of one cyclodextrin monomer with the secondary hydroxyl side of another cyclodextrin monomer, or by joining the primary hydroxyl side of one cyclodextrin monomer with the secondary hydroxyl side of another cyclodextrin monomer. Accordingly, combinations of such linkages may exist in the final copolymer. The linker group may be neutral, cationic (e.g., by containing protonated groups such as, for example, quaternary ammonium groups), or anionic (e.g., by containing deprotonated groups, such as, for example, sulfate, phosphate, borinate or carboxylate). The charge of the linker group may be adjusted by adjusting pH conditions. Examples of suitable linker groups include, but are not limited to, succinimide (e.g., dithiobis(succinimidyl propionate) DSP, and dissucinimidyl suberate (DSS)), glutamates, and aspartates).

The cyclodextrin-containing polymers which coat the paramagnetic particle of the present invention are preferably linear. As used herein, the term “linear cyclodextrin-containing polymer” refers to a polymer comprising (α, β, or γ) cyclodextrin molecules, or derivatives thereof which are inserted within a polymer chain. The term “graft polymer” as used herein refers to a polymer molecule which has additional moieties attached as pendent groups along a polymer backbone. The term “graft polymerization” denotes a polymerization in which a side chain is grafted onto a polymer chain, which side chain comprises one or several other monomers. The properties of the graft copolymer obtained such as, for example, solubility, melting point, water absorption, wettability, mechanical properties, adsorption behavior, etc., deviate more or less sharply from those of the initial polymer as a function of the type and amount of the grafted monomers. The term “grafting ratio”, as used herein, means the weight percent of the amount of the monomers grafted based on the weight of the polymer.

As used herein the terms “coat” or “coating” refers to complete or partial non-covalent association of the polymer with the surface of the paramagnetic particle.

The term “cyclodextrin moiety” refers to (α, β, or γ) cyclodextrin molecules or derivatives thereof, which may be in their oxidized or reduced forms. Cyclodextrin moieties may comprise optional linkers. Optional therapeutic agents and/or targeting ligands may be further linked to these moieties via an optional linker. The linkage may be covalent (optionally via biohydrolyzable bonds, e.g., esters, amides, carbamates, and carbonates) or may be a host-guest complex between the cyclodextrin derivative and the therapeutic agent and/or targeting ligand or the optional linkers of each. Cyclodextrin moieties may further include one or more carbohydrate moieties, preferably simple carbohydrate moieties such as galactose, attached to the cyclic core, either directly (i.e., via a carbohydrate linkage) or through a linker group.

An “effective amount” of a subject compound, with respect to the subject method of treatment, refers to an amount of the therapeutic in a preparation which, when applied as part of a desired dosage regimen provides a benefit according to clinically acceptable standards for the treatment or prophylaxis of a particular disorder.

The term “healthcare providers” refers to individuals or organizations that provide healthcare services to a person, community, etc. Examples of “healthcare providers” include doctors, hospitals, continuing care retirement communities, skilled nursing facilities, subacute care facilities, clinics, multispecialty clinics, freestanding ambulatory centers, home health agencies, and HMO's.

“Instruction(s)” as used herein means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

“Kit” as used herein means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

A “patient” or “subject” to be treated by the subject method can mean either a human or non-human subject.

The “polymerizations” of the present invention include radical, anionic, and cationic mechanisms, as well as reactions of bifunctional molecules (analogous to the formation of nylon, e.g., reacting molecules each of which bears two or more different reactive moieties that react with each other (but, preferably, are disfavored from reacting intramolecularly by steric, conformational, or other constraints), or reacting two or more different compounds, each compound bearing two or more reactive moieties that react only with reactive moieties of different compounds (i.e., intermolecularly)), as well as metal-catalyzed polymerizations such as olefin metathesis, and other polymerization reactions known to those of skill in the art.

The term “prophylacetic or therapeutic” treatment is art-recognized and includes administration to the host of one or more of the subject compositions. If it is administered prior to clinical manifestation of the unwanted condition (e.g., disease or other unwanted state of the host animal) then the treatment is prophylacetic, i.e., it protects the host against developing the unwanted condition, whereas if it is administered after manifestation of the unwanted condition, the treatment is therapeutic, (i.e., it is intended to diminish, ameliorate, or stabilize the existing unwanted condition or side effects thereof).

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylacetic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population. Prevention of pain includes, for example, reducing the frequency of, or alternatively delaying, pain sensations experienced by subjects in a treated population versus an untreated control population.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. More particularly, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used herein the term “low aqueous solubility” refers to water insoluble compounds having poor solubility in water, that is <5 mg/ml at physiological pH (6.5-7.4). Preferably, their water solubility is <1 mg/ml, more preferably <0.1 mg/ml. It is desirable that the drug is stable in water as a dispersion; otherwise a lyophilized or spray-dried solid form may be desirable.

Examples of some preferred water-insoluble drugs include immunosuppressive agents such as cyclosporins including cyclosporin (cyclosporin A), immunoactive agents, antiviral and antifungal agents, antineoplastic agents, analgesic and anti-inflammatory agents, antibiotics, anti-epileptics, anesthetics, hypnotics, sedatives, antipsychotic agents, neuroleptic agents, antidepressants, anxiolytics, anticonvulsant agents, antagonists, neuron blocking agents, anticholinergic and cholinomimetic agents, antimuscarinic and muscarinic agents, antiadrenergic and antiarrhythmics, antihypertensive agents, hormones, and nutrients. A detailed description of these and other suitable drugs may be found in Remington's Pharmaceutical Sciences, 18th edition, 1990, Mack Publishing Co. Philadelphia, Pa.

A “therapeutically effective amount” of a compound, with respect to a method of treatment, refers to an amount of the compound(s) in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

A “therapeutically effective daily dosage” of a compound, with respect to a method of treatment, refers to an amount of the compound(s) in a preparation which, when administered as part of a desired daily dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

(b) Chemical Terms

An aliphatic chain comprises the classes of alkyl, alkenyl and alkynyl defined below. A straight aliphatic chain is limited to unbranched carbon chain radicals. As used herein, the term “aliphatic group” refers to a straight chain, branched-chain, or cyclic aliphatic hydrocarbon group and includes saturated and unsaturated aliphatic groups, such as an alkyl group, an alkenyl group, and an alkynyl group.

Alkyl refers to a fully saturated branched or unbranched carbon chain radical having the number of carbon atoms specified, or up to 30 carbon atoms if no specification is made. For example, alkyl of 1 to 8 carbon atoms refers to radicals such as methyl, ethyl, propyl, butyl, pentyl, hexyl, heptyl, and octyl, and those radicals which are positional isomers of these radicals. Alkyl of 10 to 30 carbon atoms includes decyl, undecyl, dodecyl, tridecyl, tetradecyl, pentadecyl, hexadecyl, heptadecyl, octadecyl, nonadecyl, eicosyl, heneicosyl, docosyl, tricosyl and tetracosyl. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer. Likewise, preferred cycloalkyls have from 3-10 carbon atoms in their ring structure, and more preferably have 5, 6 or 7 carbons in the ring structure.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, a cyano, a nitro, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF3, —CN and the like. Exemplary substituted alkyls are described below. Cycloalkyls can be further substituted with alkyls, alkenyls, alkoxyls, alkylthios, aminoalkyls, carbonyl-substituted alkyls, —CF3, —CN, and the like.

Unless the number of carbons is otherwise specified, “lower alkyl”, as used herein, means an alkyl group, as defined above, but having from one to ten carbons, more preferably from one to six carbon atoms in its backbone structure such as methyl, ethyl, n-propyl, isopropyl, n-butyl, isobutyl, sec-butyl, and tert-butyl. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

The term “alkylthio” refers to an alkyl group, as defined above, having a sulfur radical attached thereto. In preferred embodiments, the “alkylthio” moiety is represented by one of —(S)-alkyl, —(S)-alkenyl, —(S)-alkynyl, and —(S)—(CH2)m-R1, wherein m and R1 are defined below. Representative alkylthio groups include methylthio, ethylthio, and the like.

Alkenyl refers to any branched or unbranched unsaturated carbon chain radical having the number of carbon atoms specified, or up to 26 carbon atoms if no limitation on the number of carbon atoms is specified; and having 1 or more double bonds in the radical. Alkenyl of 6 to 26 carbon atoms is exemplified by hexenyl, heptenyl, octenyl, nonenyl, decenyl, undecenyl, dodenyl, tridecenyl, tetradecenyl, pentadecenyl, hexadecenyl, heptadecenyl, octadecenyl, nonadecenyl, eicosenyl, heneicosoenyl, docosenyl, tricosenyl and tetracosenyl, in their various isomeric forms, where the unsaturated bond(s) can be located anywhere in the radical and can have either the (Z) or the (E) configuration about the double bond(s).

Alkynyl refers to hydrocarbyl radicals of the scope of alkenyl, but having 1 or more triple bonds in the radical.

The terms “alkoxyl” or “alkoxy” as used herein refers to an alkyl group, as defined below, having an oxygen radical attached thereto. Representative alkoxyl groups include methoxy, ethoxy, propoxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —O—(CH2)ₘ—R1, where m and R1 are described below.

The terms “amine” and “amino” are art-recognized and refer to both unsubstituted and substituted amines, e.g., a moiety that can be represented by the general formulae: 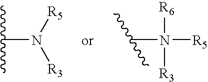
wherein R3, R5 and R6 each independently represent a hydrogen, an alkyl, an alkenyl, —(CH2)m-R1, or R3 and R5 taken together with the N atom to which they are attached complete a heterocycle having from 4 to 8 atoms in the ring structure; R1 represents an alkenyl, aryl, cycloalkyl, a cycloalkenyl, a heterocyclyl or a polycyclyl; and m is zero or an integer in the range of 1 to 8. In preferred embodiments, only one of R3 or R5 can be a carbonyl, e.g., R3, R5 and the nitrogen together do not form an imide. In even more preferred embodiments, R3 and R5 (and optionally R6) each independently represent a hydrogen, an alkyl, an alkenyl, or —(CH2)m-R1. Thus, the term “alkylamine” as used herein means an amine group, as defined above, having a substituted or unsubstituted alkyl attached thereto, i.e., at least one of R3 and R5 is an alkyl group. In certain embodiments, an amino group or an alkylamine is basic, meaning it has a pKa >7.00. The protonated forms of these functional groups have pKas relative to water above 7.00.

The term “carbonyl” is art-recognized and includes such moieties as can be represented by the general formula: 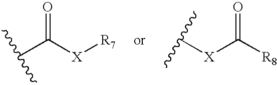
wherein X is a bond or represents an oxygen or a sulfur, and R7 represents a hydrogen, an alkyl, an alkenyl, —(CH2)m-R1 or a pharmaceutically acceptable salt, R8 represents a hydrogen, an alkyl, an alkenyl or —(CH2)m-R1, where m and R1 are as defined above. Where X is an oxygen and R7 or R8 is not hydrogen, the formula represents an “ester”. Where X is an oxygen, and R7 is as defined above, the moiety is referred to herein as a carboxyl group, and particularly when R7 is a hydrogen, the formula represents a “carboxylic acid”. Where X is an oxygen, and R8 is hydrogen, the formula represents a “formate”. In general, where the oxygen atom of the above formula is replaced by sulfur, the formula represents a “thiocarbonyl” group. Where X is a sulfur and R7 or R8 is not hydrogen, the formula represents a “thioester” group. Where X is a sulfur and R7 is hydrogen, the formula represents a “thiocarboxylic acid” group. Where X is a sulfur and R8 is hydrogen, the formula represents a “thioformate” group. On the other hand, where X is a bond, and R7 is not hydrogen, the above formula represents a “ketone” group. Where X is a bond, and R7 is hydrogen, the above formula represents an “aldehyde” group.

The term “derivatized” refers to chemically modifying molecules. The chemical modifications may be artificial such as formation of drugs, natural such as formation of metabolites. The skilled artisan would readily recognize the variety of ways molecules may be modified, such as oxidations, reductions, electrophilic/nucleophilic substitutions, alkylations, ester/amide formations and the like. For example, cyclodextrins of the present invention may be chemically modified by amination, tosylation, or iodination prior to covalently attaching them to the polymeric matrix. Likewise, therapeutic agents may be chemically modified by preparing prodrugs (e.g., glycine-camptothecin).

The terms “heterocyclyl” or “heterocyclic group” refer to 3- to 10-membered ring structures, more preferably 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, sulfamoyl, sulfinyl, ether, alkylthio, sulfonyl, ketone, aldehyde, ester, a heterocyclyl, an aromatic or heteroaromatic moiety, —CF3, —CN, or the like.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The term “hydrocarbyl” refers to a monovalent hydrocarbon radical comprised of carbon chains or rings of up to 26 carbon atoms to which hydrogen atoms are attached. The term includes alkyl, cycloalkyl, alkenyl, alkynyl and aryl groups, groups which have a mixture of saturated and unsaturated bonds, carbocyclic rings and includes combinations of such groups. It may refer to straight chain, branched-chain, cyclic structures or combinations thereof.

The term “hydrocarbylene” refers to a divalent hydrocarbyl radical. Representative examples include alkylene, phenylene, or cyclohexylene. Preferably, the hydrocarbylene chain is fully saturated and/or has a chain of 1-10 carbon atoms.

As used herein, the term “nitro” means —NO2; the term “halogen” designates —F, —Cl, —Br or —I; the term “sulfhydryl” means —SH; the term “hydroxyl” means —OH; and the term “sulfonyl” means —SO2-.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

Analogous substitutions can be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

As used herein, the definition of each expression, e.g., alkyl, m, n, etc., when it occurs more than once in any structure, is intended to be independent of its definition elsewhere in the same structure.

The terms tosyl and mesyl are art-recognized and refer to p-toluenesulfonyl and methanesulfonyl groups, respectively. The terms tosylate and mesylate are art-recognized and refer to p-toluenesulfonate ester and methanesulfonate ester, and ester functional groups and molecules that contain said groups, respectively.

Certain particles of the present invention may exist in particular geometric or stereoisomeric forms. The present invention contemplates all such compounds, including cis- and trans-isomers, (R)- and (S)-enantiomers, diastereomers, (d)-isomers, (l)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivatization with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts may be formed with an appropriate optically active acid or base, followed by resolution of the diastereomers thus formed by fractional crystallization or chromatographic means well known in the art, and subsequent recovery of the pure enantiomers.

Contemplated equivalents of the compounds described above include compounds which otherwise correspond thereto, and which have the same general properties thereof, wherein one or more simple variations of substituents are made which do not adversely affect the efficacy of the compound. In general, the particles of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

For purposes of this invention, the chemical elements are identified in accordance with the Periodic Table of the Elements, CAS version, Handbook of Chemistry and Physics, 67th Ed., 1986-87, inside cover. Also for purposes of this invention, the term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds which can be substituted or unsubstituted.

III. Exemplary Applications of Method and Compositions

(a) Exemplary Compositions

The present invention includes paramagnetic particles coated with cyclodextrin-containing polymers as contrast agents for MR imaging. In certain embodiments, one or more targeting ligands, therapeutic/bioactive agents, and/or carrier molecules are covalently attached. In certain embodiments, the surface of the paramagnetic particle is modified by inclusion guests. These inclusion guests may be further modified to comprise a targeting or therapeutic/bioactive ligand. In certain embodiments, the therapeutic agent is a small molecule, a macromolecule, an antibody, a peptide, a protein, an enzyme, a nucleic acid, or a polymer that has therapeutic function. The polymers include linear or branched cyclodextrin-containing polymers and polymers grafted with cyclodextrin. Exemplary cyclodextrin-containing polymers that may be modified as described herein are taught in U.S. Pat. No. 6,509,323, published U.S. application No. 20020151523, and U.S. Patent Application Ser. Nos. 60/417,373, and 10/372,723. These polymers are useful as carriers for small molecule therapeutic delivery, and may improve drug stability and solubility when used in vivo.

In certain embodiments, the invention relates to coated paramagnetic particles modified by inclusion complexes optionally comprising a first portion that forms an inclusion complex with the cyclodextrin moieties of the polymer, and a second portion that comprises a surface-modifying group.

Inclusion complexes are molecular compounds having the characteristic structure of an adduct, in which one of the compounds (host molecule) spatially encloses at least part of another. The enclosed compound (guest molecule) is situated in the cavity of the host molecule without affecting the framework structure of the host. It is a characteristic feature of an inclusion complex that the size and shape of the available cavity remain most often practically unaltered, apart from a slight deformation. A “host” may be any host compound or molecule known in the art. Examples of suitable “hosts” include, but are not limited to, cyclodextrins, carcerands, cavitands, crown ethers, cryptands, cucurbiturils, calixarenes, spherands, and the like. Examples of inclusion guests suitable for the complexing agents include those known in the art such as, but not limited to, adamantane, diadamantane, naphthalene, and cholesterol.

The inclusion complex may also be functionalized with surface-modifying groups that increase solubility and/or impart stabilization, particularly under biological conditions. Stabilization of the composition may be achieved or enhanced by the use of surface-modifying groups having hydrophilic groups or lipophilic groups. A preferred type of hydrophilic group is polyethylene glycol or a polyethylene glycol-containing copolymer (PEG). Preferred polyethylene ethylene glycols have the formula HO(CH₂CH₂O)_{z}—, where z varies from 2 to 500, preferably 10-300. PEG 600, PEG 3400, and PEG 5000 are representative of the polyethylene glycols which may be used in the invention. In general, the higher the molecular weight of the PEG in the surface-modifying group, the greater the stabilization of the composition. Higher molecular weight PEG's are generally preferred. A preferred surface-modifying group is pegylated adamantane or pegylated diadamantane. To increase lipophilicity (hydrophobicity), the surface-modifying group may contain lipophilic groups such as long chain alkyls, fatty acids, etc. Choice of the lipophilic group depends on the amount of lipophilicity desired. As can be seen from this discussion, the surface-modifying group may be further modified with any type of functionality to introduce a desired property into the composition. The surface-modifying group may be prepared using standard organic techniques. Employing mixtures of different surface-modifying groups allows for greater variation and specificity in achieving desired composition properties.

Cyclodextrins are a preferred host, able to interact with a great variety of ionic and molecular species and the resulting inclusion compounds belonging to the class of “host-guest” complexes. Several factors contribute to the realization of the host-guest relationship; one of them is the complementarity of the binding sites of the host and guest molecules, in the stereoelectronic sense. Cyclodextrins are capable of forming inclusion complexes with compounds having a size compatible with the dimensions of the cavity. The extent of complex formation depends, however, also on the polarity of the guest molecule. Complex formation with molecules significantly larger than the cavity may also be possible in such a way that only certain groups or side chains penetrate into the carbohydrate channel. See J. Szejtli, Akademiai Kiado, Cyclodextrins and their inclusion complexes, Budapest, 1982.

In certain embodiments, at least one polymer of the coated paramagnetic particle interacts with the inclusion guest in a host-guest interaction to form an inclusion complex between the polymer and the inclusion guest. The inclusion guest may be used to introduce functionality into a composition of the invention. In one embodiment, at least one coated paramagnetic particle has host functionality and forms an inclusion complex with an inclusion guest having guest functionality.

In one aspect of the invention, the surface-modifying group stabilizes the coated paramagnetic complex. In certain embodiments, the surface-modifying group is a stabilizing group, selected from phosphate, diphosphate, carboxylate, polyphosphate, thiophosphate, phosphonate, thiophosphonate, sulphate, sulphonate, mercapto, silanetriol, trialkoxysilane-containing polyalkylene glycols, carbohydrates or phosphate-containing nucleotides, the oligomers thereof or the polymers thereof. In certain embodiments, the surface-modifying group further comprises a ligand, such as targeting ligands and/or therapeutic agents.

Accordingly, one embodiment of the invention is a paramagnetic particle coated by a cyclodextrin-containing polymeric compound wherein the cyclodextrin polymer comprises n′ units of U, wherein n′ represents an integer in the range of 1 to about 30,000, and U is represented by the general formula: 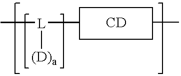
wherein,

CD represents a cyclodextrin molecule, or derivative thereof;

L represents a linker group;

D, independently for each occurrence, represents a targeting ligand, a therapeutic agent or prodrug thereof, or a carrier ligand;

a, independently for each occurrence, represents an integer in the range of 0 and 10 (preferably 1 to 8, 1 to 5, or even 1 to 3).

In certain embodiments, n′ is an integer in the range 3-100, 5-100, or even 10-100 or 15-100.

In certain embodiments, the surface of the polymer is modified by one or more inclusion guests, each inclusion guest comprising a first portion that forms an inclusion complex with the cyclodextrin moieties of the polymer, and a second portion that comprises a surface-modifying group.

In preferred embodiments, the paramagnetic particle is a superparamagnetic particle.

In certain embodiments, the paramagnetic particle comprises a metal oxide. In particular embodiments, the metal oxide is an oxide of cobalt, manganese, or iron. In certain embodiments, the paramagnetic particle has an average hydrodynamic diameter in the range of 30-90 nanometers.

In certain embodiments, the underlying polymers are linear cyclodextrin-containing polymers, e.g., the polymer backbone includes cyclodextrin moieties. For example, the polymer may be a water-soluble, linear cyclodextrin polymer, e.g., capable of being produced by providing at least one cyclodextrin derivative modified to bear one reactive site at each of exactly two positions, and reacting the cyclodextrin derivative with a linker having exactly two reactive moieties capable of forming a covalent bond with the reactive sites under polymerization conditions that promote reaction of the reactive sites with the reactive moieties to form covalent bonds between the linker and the cyclodextrin derivative, whereby a linear polymer comprising alternating units of cyclodextrin derivatives and linkers is produced. Alternatively the polymer may be a water-soluble, linear cyclodextrin polymer having a linear polymer backbone, which polymer comprises a plurality of substituted or unsubstituted cyclodextrin moieties and linker moieties in the linear polymer backbone, wherein each of the cyclodextrin moieties, other than a cyclodextrin moiety at the terminus of a polymer chain, is attached to two of said linker moieties, each linker moiety, other than a linker moiety at the terminus of a polymer chain, covalently linking two cyclodextrin moieties. In yet another embodiment, the polymer is a water-soluble, linear cyclodextrin polymer comprising a plurality of cyclodextrin moieties covalently linked together by a plurality of linker moieties, wherein each cyclodextrin moiety, other than a cyclodextrin moiety at the terminus of a polymer chain, is attached to two linker moieties to form a linear cyclodextrin polymer.

The linker group(s) may be an alkylene chain, a polyethylene glycol (PEG) chain, polysuccinic anhydride, poly-L-glutamic acid, poly(ethyleneimine), an oligosaccharide, an amino acid chain, or any other suitable linkage. In certain embodiments, the linker group itself can be stable under physiological conditions, such as an alkylene chain, or it can be cleavable under physiological conditions, such as by an enzyme (e.g., the linkage contains a peptide sequence that is a substrate for a peptidase), or by hydrolysis (e.g., the linkage contains a hydrolyzable group, such as an ester or thioester). The linker groups can be biologically inactive, such as a PEG, polyglycolic acid, or polylactic acid chain, or can be biologically active, such as an oligo- or polypeptide that, when cleaved from the moieties, binds a receptor, deactivates an enzyme, etc. Various oligomeric linker groups that are biologically compatible and/or bioerodible are known in the art, and the selection of the linkage may influence the ultimate properties of the material, such as whether it is durable when implanted, whether it gradually deforms or shrinks after implantation, or whether it gradually degrades and is absorbed by the body. The linker group may be attached to the moieties by any suitable bond or functional group, including carbon-carbon bonds, esters, ethers, amides, amines, carbonates, carbamates, sulfonamides, etc.

In certain embodiments, the linker group(s) of the present invention an alkylene group wherein one or more methylene groups is optionally replaced by a group Y, wherein each Y, independently for each occurrence, is selected from substituted or unsubstituted aryl, heteroaryl, cycloalkyl, heterocycloalkyl, or —O—, C(═X) (wherein X is NR₁, O or S), —OC(O)—, —C(═O)O, —NR₁—, —NR₁CO—, —C(O)NR₁—, —S(O)ₙ— (wherein n is 0, 1, or 2), —OC(O)—NR₁, —NR₁—C(O)—NR₁—, —NR₁—C(NR₁)—NR₁—, and —B(OR₁)— (wherein R₁, independently for each occurrence, represents H or a lower alkyl), selected such that no two heteroatoms are directly adjacent to each other.

In certain embodiments, the linker group represents a derivatized or non-derivatized amino acid. In certain embodiments, linker groups with one or more terminal carboxyl groups may be conjugated to the polymer. In certain embodiments, one or more of these terminal carboxyl groups may be capped by covalently attaching them to a therapeutic agent, a targeting moiety, or a cyclodextrin moiety via an (thio)ester or amide bond. In still other embodiments, linker groups with one or more terminal hydroxyl, thiol, or amino groups may be incorporated into the polymer. In preferred embodiments, one or more of these terminal hydroxyl groups may be capped by covalently attaching them to a therapeutic agent, a targeting moiety, or a cyclodextrin moiety via an (thio)ester, amide, carbonate, carbamate, thiocarbonate, or thiocarbamate bond. In certain embodiments, these (thio)ester, amide, (thio)carbonate or (thio)carbamates bonds may be biohydrolyzable, i.e., capable of being hydrolyzed under biological conditions.

In certain embodiments, the polymers as described above have polydispersities less than about 3, or even less than about 2.

In certain embodiments, the therapeutic agent is a small molecule, a peptide, a protein, or a polymer that has therapeutic function. In certain embodiments, the agent is an anti-cancer (such as camptothecin or related derivatives), anti-fungal, anti-bacterial, anti-mycotic, or anti-viral therapeutic. In certain embodiments, the agent is a receptor agonist. In certain embodiments, the agent is a receptor antagonist. In certain embodiments, the therapeutic agent is a protease inhibitor. Furthermore, a polymer of the present invention may contain one kind of therapeutic agent, or may contain more than one kind of therapeutic agent. For instance, two or more different cancer drugs, or a cancer drug and an immunosuppressant, or an antibiotic and an anti-inflammatory agent may be grafted on to the polymer via optional linkers. By selecting different linkers for different drugs, the release of each drug may be attenuated to achieve maximal dosage and efficacy.

One embodiment of the present invention provides an improved delivery of certain hydrophobic small molecule therapeutics by covalently conjugating them to cyclodextrin containing polymers. Such conjugation improves the aqueous solubility and hence the bioavailability of the therapeutic agents. Accordingly, in one embodiment of the invention, the therapeutic agent is a hydrophobic compound with a log P>0.4, >0.6, >0.8, >1, >2, >3, >4, or even >5. In other embodiments, a hydrophobic therapeutic agent, such as camptothecin, may be conjugated to another compound, such as an amino acid, prior to covalently attaching the conjugate on to the polymer.

In certain embodiments, the cyclodextrin moieties make up at least about 2%, 5% or 10% by weight, up to 20%, 30%, 50% or even 80% of the cyclodextrin-modified polymer by weight. In certain embodiments, the therapeutic agents, or targeting ligands make up at least about 1%, 5%, 10% or 15%, 20%, 25%, 30% or even 35% of the cyclodextrin-modified polymer by weight. Number-average molecular weight (Mₙ) may also vary widely, but generally fall in the range of about 1,000 to about 500,000 daltons, preferably from about 5000 to about 200,000 daltons and, even more preferably, from about 10,000 to about 100,000. Most preferably, Mₙ varies between about 12,000 and 65,000 daltons. In certain other embodiments, Mₙ varies between about 3000 and 150,000 daltons. Within a given sample of a subject polymer, a wide range of molecular weights may be present. For example, molecules within the sample may have molecular weights that differ by a factor of 2, 5, 10, 20, 50, 100, or more, or that differ from the average molecular weight by a factor of 2, 5, 10, 20, 50, 100, or more. Exemplary cyclodextrin moieties include cyclic structures consisting essentially of from 7 to 9 saccharide moieties, such as cyclodextrin and oxidized cyclodextrin. A cyclodextrin moiety optionally comprises a linker moiety that forms a covalent linkage between the cyclic structure and the polymer backbone, preferably having from 1 to 20 atoms in the chain, such as alkyl chains, including dicarboxylic acid derivatives (such as glutaric acid derivatives, succinic acid derivatives, and the like), and heteroalkyl chains, such as oligoethylene glycol chains.

Cyclodextrins are cyclic polysaccharides containing naturally occurring D-(+)-glucopyranose units in an α-(1,4) linkage. The most common cyclodextrins are alpha (α)-cyclodextrins, beta (β)-cyclodextrins and gamma (γ)-cyclodextrins which contain, respectively six, seven, or eight glucopyranose units. Structurally, the cyclic nature of a cyclodextrin forms a torus or donut-like shape having an inner apolar or hydrophobic cavity, the secondary hydroxyl groups situated on one side of the cyclodextrin torus and the primary hydroxyl groups situated on the other. Thus, using (β)-cyclodextrin as an example, a cyclodextrin is often represented schematically as follows.

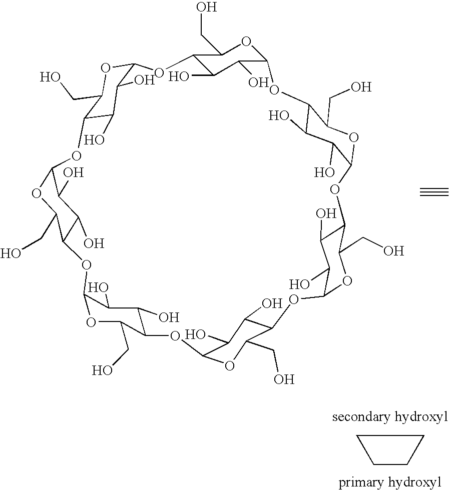

The side on which the secondary hydroxyl groups are located has a wider diameter than the side on which the primary hydroxyl groups are located. The present invention contemplates covalent linkages to cyclodextrin moieties on the primary and/or secondary hydroxyl groups. The hydrophobic nature of the cyclodextrin inner cavity allows for host-guest inclusion complexes of a variety of compounds, e.g., adamantane. (Comprehensive Supramolecular Chemistry, Volume 3, J. L. Atwood et al., eds., Pergamon Press (1996); T. Cserhati, Analytical Biochemistry, 225:328-332(1995); Husain et al., Applied Spectroscopy, 46:652-658 (1992); FR 2 665 169). Additional methods for modifying polymers are disclosed in Suh, J. and Noh, Y., Bioorg. Med. Chem. Lett. 1998, 8, 1327-1330.

In certain embodiments, the present invention contemplates a paramagnetic particle coated by a linear, water-soluble, cyclodextrin-containing polymer, wherein a plurality of bioactive moieties are covalently attached to the polymer through attachments that are cleaved under biological conditions to release the bioactive moieties. In certain embodiments, the surface of the paramagnetic particle is modified by inclusion guests. These inclusion guests may comprise a targeting or therapeutic/bioactive moiety. In all cases, administration of the polymer to a patient results in release of the bioactive agent over a period of at least 2, 3, 5, 6, 8, 10, 15, 20, 24, 36, 48 or even 72 hours.

In certain embodiments, the present invention contemplates attenuating the rate of release of the therapeutic agent by introducing various linking groups between the therapeutic agent and/or targeting ligand and the polymer-coated particle. Thus, in certain embodiments, the polymer-coated particles of the present invention are compositions for controlled delivery of therapeutic agents.

In other embodiments, the coated paramagnetic particles stabilize the bioactive form of a therapeutic agent which exists in equilibrium between an active and inactive form. For instance, conjugating the therapeutic agent to the coated paramagnetic particles of the present invention may shift the equilibrium between two tautomeric forms of the agent to the bioactive tautomer. In other embodiments, the coated paramagnetic particles may modulate the equilibrium between lactonic and acid forms of a therapeutic agent.

One aspect of the present invention contemplates attaching a hydrophobic therapeutic agent such as (S)-20-camptothecin to linear or branched polymer-coated particles for better delivery of the drug. (S)-20-camptothecin (CPT), an alkaloid isolated from Camptitheca accuminata in the late 1950's, was found to exhibit anticancer activity by inhibiting the action of topoisomerase I during the S-phase of the cell cycle. Its application in human cancer treatment, however, is limited due to several factors, especially its undesirable interactions with human serum albumin, instability of the bioactive lactone form, and poor aqueous solubility. In order to circumvent this problem, many CPT analogs have been developed to improve lactone stability and aqueous solubility. Topotecan and irinotecan are analogs of CPT that have already been approved by FDA for human cancer treatment. The present invention discloses paramagnetic particles coated with various types of linear, branched, or grafted cyclodextrin-containing polymers wherein (S)-20-camptothecin is covalently bound to the polymer. In certain embodiments, the drug is covalently linked via a biohydrolyzable bond selected from an ester, amide, carbamates, or carbonate.

An exemplary synthetic scheme for covalently bonding a derivatized CD to 20(S)-camptothecin is shown in Scheme I. 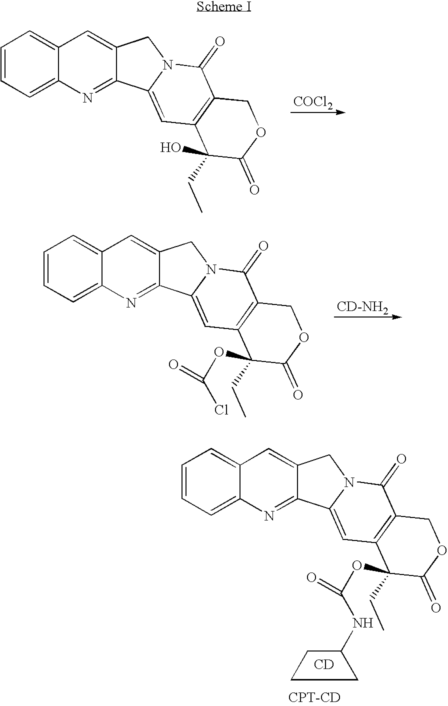
Examples of different ways of synthesizing exemplary linear cyclodextrin-CPT polymers are described in detail in U.S. Publication No. 20040077595.
(b) Targeting Ligand

As mentioned above, one aspect of the present invention contemplates attaching a therapeutic agent to the coated paramagnetic particles described herein.

In certain embodiments, the coated paramagnetic particle further comprises a targeting ligand. Thus in certain embodiments, a receptor, cell, and/or tissue-targeting ligand, or a precursor thereof is coupled to a polymer conjugate. As used herein the term “targeting ligand” refers to any material or substance which may promote targeting of receptors, cells, and/or tissues in vivo or in vitro with the coated particles of the present invention. The targeting ligand may be synthetic, semi-synthetic, or naturally-occurring. Materials or substances which may serve as targeting ligands include, for example, proteins, including antibodies, antibody fragments, hormones, hormone analogues, glycoproteins and lectins, peptides, polypeptides, amino acids, sugars, saccharides, including monosaccharides and polysaccharides, carbohydrates, small molecules, vitamins, steroids, steroid analogs, hormones, cofactors, bioactive agents, and genetic material, including nucleosides, nucleotides, nucleotide acid constructs and polynucleotides. As used herein, the term “precursor” to a targeting ligand refers to any material or substance which may be converted to a targeting ligand. Such conversion may involve, for example, anchoring a precursor to a targeting ligand. Exemplary targeting precursor moieties include maleimide groups, disulfide groups, such as ortho-pyridyl disulfide, vinylsulfone groups, azide groups, and α-iodo acetyl groups. The attachment of the targeting ligand or precursor thereof to the polymer may be accomplished in various ways including but not limited to chelation, covalent attachment, or formation of host-guest complexes. In certain embodiments, an optional linker group may be present between the targeting ligand or precursor thereof and the polymer, wherein the linker group is attached to the polymer via chelation, covalent attachment or host-guest complexes. For example, the one terminal end of a linker group may be attached to the targeting ligand while the other may be attached to an adamantane group, or other such hydrophobic moiety, which forms a host guest complex with a cyclodextrin moiety. Thus the targeting ligand may be attached to a grafted cyclodextrin moiety, to a cyclodextrin moiety within the polymeric chain, or to the polymeric chain itself. The number of targeting ligands per polymeric chain may vary according to various factors including but not limited to the identity of the therapeutic agent, nature of the disease, type of polymer chain. Structures of possible linker groups are the same as linker groups defined elsewhere in this application.

(c) Pharmaceutical Compositions, Formulations and Dosages

In part, a biocompatible polymer-coated paramagnetic particle composition of the present invention includes a biocompatible and optionally biodegradable polymer, such as one having recurring monomeric units, optionally including any other biocompatible and optionally biodegradable polymer mentioned above or known in the art. In certain embodiments, the compositions are non-pyrogenic, e.g., do not trigger elevation of a patient's body temperature by more than a clinically acceptable amount.

The subject compositions may contain a “drug,” “therapeutic agent,” “medicament,” or “bioactive substance,” which are biologically, physiologically, or pharmacologically active substances that act locally or systemically in the human or animal body. For example, a subject composition may include any of the other compounds discussed above.

Various forms of the medicaments or biologically active materials may be used which are capable of being released from the coated polymer into adjacent tissues or fluids. They may be hydrophobic molecules, neutral molecules, polar molecules, or molecular complexes capable of hydrogen bonding. They may be in the form of ethers, esters, amides and the like, including prodrugs which are biologically activated when injected into the human or animal body, e.g., by cleavage of an ester or amide. A therapeutic agent in a subject composition may vary widely with the purpose for the composition.

Plasticizers and stabilizing agents known in the art may be incorporated in polymers of the present invention. In certain embodiments, additives such as plasticizers and stabilizing agents are selected for their biocompatibility. In certain embodiments, the additives are lung surfactants, such as 1,2-dipalmitoylphosphatidycholine (DPPC) and L-α-phosphatidylcholine (PC).

A composition of this invention may further contain one or more adjuvant substances, such as fillers, thickening agents or the like. In other embodiments, materials that serve as adjuvants may be associated with the polymer matrix. Such additional materials may affect the characteristics of the polymer matrix that results.

For example, fillers, such as bovine serum albumin (BSA) or mouse serum albumin (MSA), may be associated with the polymer matrix. In certain embodiments, the amount of filler may range from about 0.1 to about 50% or more by weight of the polymer matrix, or about 2.5, 5, 10, 25, or 40 percent. Incorporation of such fillers may affect the biodegradation of the polymeric material and/or the sustained release rate of any encapsulated substance. Other fillers known to those of skill in the art, such as carbohydrates, sugars, starches, saccharides, celluloses and polysaccharides, including mannitose and sucrose, may be used in certain embodiments of the present invention.

In certain embodiments, the coated paramagnetic particle includes an excipient. A particular excipient may be selected based on its melting point, solubility in a selected solvent (e.g., a solvent that dissolves the polymer and/or the therapeutic agent), and the resulting characteristics of the microparticles.

Excipients may make up a few percent, about 5%, 10%, 15%, 20%, 25%, 30%, 40%, 50%, or higher percentage of the subject compositions.

Buffers, acids and bases may be incorporated in the subject compositions to adjust their pH. Agents to increase the diffusion distance of agents released from the polymer matrix may also be included.

The charge, lipophilicity or hydrophilicity of any subject polymeric matrix may be modified by attaching in some fashion an appropriate compound to the surface of the matrix. For example, surfactants may be used to enhance wettability of poorly soluble or hydrophobic compositions. Examples of suitable surfactants include dextran, polysorbates and sodium lauryl sulfate. In general, surfactants are used in low concentrations, generally less than about 5%.

Binders are adhesive materials that may be incorporated in polymeric formulations to bind and maintain matrix integrity. Binders may be added as dry powder or as solution. Sugars and natural and synthetic polymers may act as binders.

Materials added specifically as binders are generally included in the range of about 0.5%-15% w/w of the matrix formulation. Certain materials, such as microcrystalline cellulose, also used as a spheronization enhancer, also have additional binding properties.

Various coatings may be applied to modify the properties of the matrices.

Three exemplary types of coatings are seal, gloss and enteric coatings. Other types of coatings having various dissolution or erosion properties may be used to further modify subject matrices behavior, and such coatings are readily known to one of ordinary skill in the art.

The seal coat may prevent excess moisture uptake by the matrices during the application of aqueous based enteric coatings. The gloss coat generally improves the handling of the finished matrices. Water-soluble materials such as hydroxypropylcellulose may be used to seal coat and gloss coat implants. The seal coat and gloss coat are generally sprayed onto the matrices until an increase in weight between about 0.5% and about 5%, often about 1% for a seal coat and about 3% for a gloss coat, has been obtained.

Enteric coatings consist of polymers which are insoluble in the low pH (less than 3.0) of the stomach, but are soluble in the elevated pH (greater than 4.0) of the small intestine. Polymers such as EUDRAGIT™, RohmTech, Inc., Malden, Mass., and AQUATERIC™, FMC Corp., Philadelphia, Pa., may be used and are layered as thin membranes onto the implants from aqueous solution or suspension or by a spray drying method. The enteric coat is generally sprayed to a weight increase of about 1% to about 30%, preferably about 10 to about 15% and may contain coating adjuvants such as plasticizers, surfactants, separating agents that reduce the tackiness of the implants during coating, and coating permeability adjusters.

The precise time of administration and/or amount of therapeutic polymer-coated particles that will yield the most effective results in terms of efficacy of treatment in a given patient will depend upon the activity, pharmacokinetics, and bioavailability of a particular compound, physiological condition of the patient (including age, sex, disease type and stage, general physical condition, responsiveness to a given dosage and type of medication), route of administration, etc. However, the above guidelines can be used as the basis for fine-tuning the treatment, e.g., determining the optimum time and/or amount of administration, which will require no more than routine experimentation, such as monitoring the subject and adjusting the dosage and/or timing.

The phrase “pharmaceutically acceptable” is employed herein to refer to those materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically acceptable salts” refers to the relatively non-toxic, inorganic and organic acid addition salts of the polymer-coated particles. These salts can be prepared in situ during the final isolation and purification of the polymer-coated particles, or by separately reacting a purified polymer in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts and the like. (See, for example, Berge et al. (1977) “Pharmaceutical Salts”, J. Pharm. Sci. 66:1-19)

In other cases, the therapeutic polymer-coated particles useful in the methods of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of the polymer(s). These salts can likewise be prepared in situ during the final isolation and purification of the polymer(s), or by separately reacting the purified polymer(s) in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the polymer-coated particles.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a polymer-coated particle with the carrier and, optionally, one or more accessory ingredients. In general, the formulations are prepared by uniformly and intimately bringing into association a polymer-coated particle with liquid carriers, or finely divided solid carriers, or both, and then, if necessary, shaping the product.

Formulations suitable for oral administration may be in the form of capsules, cachets, pills, tablets, powders, granules, or as a solution or a suspension in an aqueous or non-aqueous liquid, or as an oil-in-water or water-in-oil liquid emulsion, or as an elixir or syrup, each containing a predetermined amount of a polymer-coated particle as an active ingredient. A composition may also be administered as a bolus, electuary or paste.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the polymer-coated particles, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Polymer-coated particles of this invention suitable for parenteral administration may be formulated with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the polymer-coated particle compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

When the polymer-coated particle(s) of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The preparations of agents may be given orally or parenterally. They are of course given by forms suitable for each administration route. For example, they are administered in tablets or capsule form, or by injection, inhalation, or infusion. Injection or infusion is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a therapeutic polymer conjugate, drug or other material other than directly into the central nervous system, such that it enters the patient's system and, thus, is subject to metabolism and other like processes, for example, subcutaneous administration.

These polymer-coated particle(s) may be administered to humans and other animals for therapy by any suitable route of administration, including orally, nasally, as by, for example, a spray, parenterally, and intracisternally.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

(d) General Procedure for Preparation of Polymer-Coated Paramagnetic Particle

It is to be understood throughout this detailed description, that the use of superparamagnetic iron oxides as NMR contrast agents is but one embodiment of the invention and that superparamagnetic oxides of other magnetic metals, e.g., cobalt or gadolinium, may be substituted for iron oxides without departing from the spirit and scope of the invention.

There are two general strategies for the formation of the coated superparamagnetic iron oxide particles suitable for MRI.

1. Synthesis of iron oxide by precipitation in the presence of carbohydrate polymers. Such syntheses include those described by London et al., U.S. Pat. No. 2,870,740, Molday, U.S. Pat. No. 4,452,773, Cox et al., Nature, 208, 237 (1965) and Rembaum, U.S. Pat. No. 4,267,234; all of which are incorporated herein by reference.

2. Synthesis of the iron oxide by precipitation followed by coating with a carbohydrate polymer. This type of synthetic route is utilized by Elmore, Phys. Rev. 54, 309 (1938) and Ohgushi et al., J. Mag Res., 29, 599 (1978); both of which are incorporated herein by reference.

With carbohydrates, such as dextrans, synthesis of the oxide in the presence of the polymer seems to effect a tight association between the polymer and the oxide. The association need only be strong enough such that the oxide and adsorbed polymer can be manipulated, stored and injected in the presence of nonadsorbed polymer, if necessary. Coating methods are general and can be readily adapted for use in the preparation of the subject coated particles, particularly when using polymers with molecular weights from about 5,000 to about 250,000 daltons.

(e) Biodegradability and Release Characteristics

In certain embodiments, the polymer-coated particle(s) of the present invention, upon contact with body fluids, undergo gradual degradation. The life of a polymer-coated particle in vivo depends upon, among other things, its molecular weight, crystallinity, biostability, and the degree of crosslinking. In general, the greater the molecular weight, the higher the degree of crystallinity, and the greater the biostability, the slower biodegradation will be.

If a subject composition is formulated with a therapeutic agent or other material, release of such an agent or other material for a sustained or extended period as compared to the release from an isotonic saline solution generally results. Such release profile may result in prolonged delivery (over, say 1 to about 2,000 hours, or alternatively about 2 to about 800 hours) of effective amounts (e.g., about 0.0001 mg/kg/hour to about 1 0 mg/kg/hour) of the agent or any other material associated with the polymer.

One protocol generally accepted in the field that may be used to determine the release rate of any therapeutic agent or other material loaded in the polymer-coated particle of the present invention involves degradation of any such matrix in a 0.1 M PBS solution (pH 7.4) at 37° C., an assay known in the art. For purposes of the present invention, the term “PBS protocol” is used herein to refer to such protocol.

In certain instances, the release rates of different polymer systems of the present invention may be compared by subjecting them to such a protocol. Release of any material incorporated into or grafted onto the polymer matrix may be characterized in certain instances by an initial increased release rate, which may release from about 5 to about 50% or more of any incorporated material, or alternatively about 10, 15, 20, 25, 30 or 40%, followed by a release rate of lesser magnitude.

The release rate of any therapeutic material may also be characterized by the amount of such material released per day per mg of polymer matrix. For example, in certain embodiments, the release rate may vary from about 1 ng or less of any therapeutic material per day per mg of polymeric system to about 500 or more ng/day/mg. Alternatively, the release rate may be about 0.05, 0.5, 5, 10, 25, 50, 75, 100, 125, 150, 175, 200, 250, 300, 350, 400, 450, or 500 ng/day/mg. In still other embodiments, the release rate of any therapeutic material may be 10,000 ng/day/mg, or even higher.

In addition to the embodiment involving protocols for in vitro determination of release rates, in vivo protocols, whereby in certain instances release rates for polymeric systems may be determined in vivo, are also contemplated by the present invention. Other assays useful for determining the release of any material from the polymers of the present system are known in the art.

(f) Methods of Manufacturing Polymers that Coat Paramagnetic Particles

Generally, functionalized polymers for coating particles of the present invention can be prepared in one of two ways: monomers bearing therapeutic agents, targeting ligands, and/or cyclodextrin moieties can be polymerized, or polymer backbones can be derivatized with therapeutic agents, targeting ligands, and/or cyclodextrin moieties.

For example, if the polymer includes alcohols, thiols, or amines as reactive groups, the grafting agents may include reactive groups that react with them, such as isocyanates, isothiocyanates, acid chlorides, acid anhydrides, epoxides, ketenes, sulfonyl chlorides, activated carboxylic acids (e.g., carboxylic acids treated with an activating agent such as PyBrOP, carbonyldiimidazole, or another reagent that reacts with a carboxylic acid to form a moiety susceptible to nucleophilic attack), or other electrophilic moieties known to those of skill in the art. In certain embodiments, a catalyst may be needed to cause the reaction to take place (e.g., a Lewis acid, a transition metal catalyst, an amine base, etc.) as will be understood by those of skill in the art.

In certain embodiments, the different grafting agents are reacted with the polymer simultaneously or substantially simultaneously (e.g., in a one-pot reaction), or are reacted sequentially with the polymer (optionally with a purification and/or wash step between reactions).

Another aspect of the polymers is a method for manufacturing linear or branched cyclodextrin-containing polymers. While the discussion below focuses on the preparation of linear cyclodextrin molecules, one skilled in the art would readily recognize that the methods described can be adapted for producing branched polymers by choosing an appropriate comonomer A precursor.

Accordingly, one embodiment of the polymers is a method of preparing a linear cyclodextrin copolymer. A linear cyclodextrin copolymer may be prepared by copolymerizing a cyclodextrin monomer precursor disubstituted with an appropriate leaving group with a comonomer A precursor capable of displacing the leaving groups. The leaving group, which may be the same or different, may be any leaving group known in the art which may be displaced upon copolymerization with a comonomer A precursor. In a preferred embodiment, a linear cyclodextrin copolymer may be prepared by iodinating a cyclodextrin monomer precursor to form a diiodinated cyclodextrin monomer precursor and copolymerizing the diiodinated cyclodextrin monomer precursor with a comonomer A precursor to form a linear cyclodextrin copolymer having a repeating unit of formula II or III, or a combination thereof, each as described above. While examples presented below discuss iodinated cyclodextrin moieties, one skilled in the art would readily recognize the suitability of cyclodextrin moieties wherein other leaving groups such as alkyl and aryl sulfonate may be present instead of iodo groups. For example, one method of preparing a linear cyclodextrin copolymer involves iodinating a cyclodextrin monomer precursor as described above to form a diiodinated cyclodextrin monomer precursor of formula Iva, IVb, IVc or a mixture thereof:

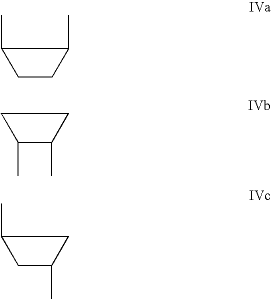

The diiodinated cyclodextrin may be prepared by any means known in the art. (Tabushi et al. J. Am. Chem. 106, 5267-5270 (1984); Tabushi et al. J. Am. Chem. 106, 4580-4584 (1984)). For example, β-cyclodextrin may be reacted with biphenyl-4,4′-disulfonyl chloride in the presence of anhydrous pyridine to form a biphenyl-4,4′-disulfonyl chloride capped β-cyclodextrin which may then be reacted with potassium iodide to produce diiodo-β-cyclodextrin. The cyclodextrin monomer precursor is iodinated at only two positions. By copolymerizing the diiodinated cyclodextrin monomer precursor with a comonomer A precursor, as described above, a linear cyclodextrin polymer having a repeating unit of Formula Ia, Ib, or a combination thereof, also as described above, may be prepared. If appropriate, the iodine or iodo groups may be replaced with other known leaving groups.

Also, the iodo groups or other appropriate leaving group may be displaced with a group that permits reaction with a comonomer A precursor, as described above. For example, a diiodinated cyclodextrin monomer precursor of formula IVa, IVb, IVc or a mixture thereof may be aminated to form a diaminated cyclodextrin monomer precursor of formula Va, Vb, Vc or a mixture thereof:

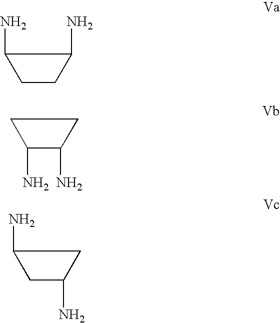

The diaminated cyclodextrin monomer precursor may be prepared by any means known in the art. (Tabushi et al. Tetrahedron Lett. 18:11527-1530 (1977); Mungall et al., J. Org. Chem. 16591662 (1975)). For example, a diiodo-β-cyclodextrin may be reacted with sodium azide and then reduced to form a diamino-β-cyclodextrin). The cyclodextrin monomer precursor is aminated at only two positions. The diaminated cyclodextrin monomer precursor may then be copolymerized with a comonomer A precursor, as described above, to produce a linear cyclodextrin copolymer having a repeating unit of formula II-III or a combination thereof, also as described above. However, the amino functionality of a diaminated cyclodextrin monomer precursor need not be directly attached to the cyclodextrin moiety. Alternatively, the amino functionality or another nucleophilic functionality may be introduced by displacement of the iodo or other appropriate leaving groups of a cyclodextrin monomer precursor with amino group containing moieties such as, for example, HSCH₂CH₂NH₂ (or a di-nucleophilic molecule more generally represented by HW—(CR₁R₂)ₙ— WH wherein W, independently for each occurrence, represents O, S, or NR₁; R₁ and R₂, independently for each occurrence, represent H, (un)substituted alkyl, (un)substituted aryl, (un)substituted heteroalkyl, (un)substituted heteroaryl) with an appropriate base such as a metal hydride, alkali or alkaline carbonate, or tertiary amine to form a diaminated cyclodextrin monomer precursor of formula Vd, Ve, Vf or a mixture thereof:

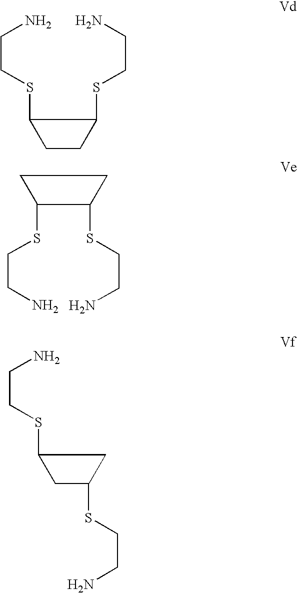

A linear oxidized cyclodextrin-containing copolymer may be prepared by oxidizing a linear cyclodextrin-containing copolymer as described below. This method may be performed as long as the comonomer A does not contain an oxidation-sensitive moiety or group such as, for example, a thiol.

A linear cyclodextrin copolymer may be oxidized so as to introduce at least one oxidized cyclodextrin monomer into the copolymer such that the oxidized cyclodextrin monomer is an integral part of the polymer backbone. A linear cyclodextrin copolymer which contains at least one oxidized cyclodextrin monomer is defined as a linear oxidized cyclodextrin copolymer or a linear oxidized cyclodextrin-containing polymer. The cyclodextrin monomer may be oxidized on either the secondary or primary hydroxyl side of the cyclodextrin moiety. If more than one oxidized cyclodextrin monomer is present in a linear oxidized cyclodextrin copolymer, the same or different cyclodextrin monomers oxidized on either the primary hydroxyl side, the secondary hydroxyl side, or both may be present. Oxidized cyclodextrins are discussed in detail in U.S. Pat. Nos. 6,509,323, 6,884,789 and 7,091,192, and U.S. application Ser. No. 11/358,976, which are hereby incorporated by reference.

A linear oxidized cyclodextrin copolymer may be prepared by oxidation of a linear cyclodextrin copolymer. Oxidation of a linear cyclodextrin copolymer may be accomplished by oxidation techniques known in the art. (Hisamatsu et al., Starch 44:188-191 (1992)). Preferably, an oxidant such as, for example, sodium periodate is used. It would be understood by one of ordinary skill in the art that under standard oxidation conditions the degree of oxidation may vary or be varied per copolymer. Thus, a linear oxidized copolymer may contain one oxidized cyclodextrin monomer. In another embodiment, substantially all cyclodextrin monomers of the copolymer would be oxidized.

Another method of preparing a linear oxidized cyclodextrin copolymer involves the oxidation of a diiodinated or diaminated cyclodextrin monomer precursor to form an oxidized diiodinated or diaminated cyclodextrin monomer precursor and copolymerization of the oxidized diiodinated or diaminated cyclodextrin monomer precursor with a comonomer A precursor.

Alternatively, an oxidized diiodinated or diaminated cyclodextrin monomer precursor may be prepared by oxidizing a cyclodextrin monomer precursor to form an oxidized cyclodextrin monomer precursor and then diiodinating and/or diaminating the oxidized cyclodextrin monomer. The cyclodextrin moiety may be modified with other leaving groups other than iodo groups and other amino group containing functionalities. The oxidized diiodinated or diaminated cyclodextrin monomer precursor may then be copolymerized with a comonomer A precursor to form a linear oxidized cyclodextrin copolymer.

A linear oxidized cyclodextrin copolymer may also be further modified by attachment of at least one ligand to the copolymer. The ligand is as described above.

A linear cyclodextrin copolymer or linear oxidized cyclodextrin copolymer may be attached to or grafted onto a substrate. The substrate may be any substrate as recognized by those of ordinary skill in the art. A linear cyclodextrin copolymer or linear oxidized cyclodextrin copolymer may be crosslinked to a polymer to form, respectively, a crosslinked cyclodextrin copolymer or a crosslinked oxidized cyclodextrin copolymer. The polymer may be any polymer capable of crosslinking with a linear or linear oxidized cyclodextrin copolymer (e.g., polyethylene glycol (PEG) polymer, polyethylene polymer). The polymer may also be the same or different linear cyclodextrin copolymer or linear oxidized cyclodextrin copolymer. Thus; for example, a linear cyclodextrin copolymer may be crosslinked to any polymer including, but not limited to, itself, another linear cyclodextrin copolymer, and a linear oxidized cyclodextrin copolymer. A crosslinked linear cyclodextrin copolymer may be prepared by reacting a linear cyclodextrin copolymer with a polymer in the presence of a crosslinking agent. A crosslinked linear oxidized cyclodextrin copolymer may be prepared by reacting a linear oxidized cyclodextrin copolymer with a polymer in the presence of an appropriate crosslinking agent. The crosslinking agent may be any crosslinking agent known in the art. Examples of crosslinking agents include dihydrazides and disulfides. In a preferred embodiment, the crosslinking agent is a labile group such that a crosslinked copolymer may be uncrosslinked if desired.

A cyclodextrin composition containing at least one linear cyclodextrin copolymer and at least one linear oxidized cyclodextrin copolymer may also be employed. Accordingly, either or both of the linear cyclodextrin copolymer and linear oxidized cyclodextrin copolymer may be crosslinked to another polymer and/or bound to a ligand as described above. A linear cyclodextrin copolymer, a linear oxidized cyclodextrin copolymer and their crosslinked derivatives are as described above. A therapeutic agent covalently or non-covalently coupled to the polymer may be any synthetic or naturally occurring biologically active therapeutic agent including those known in the art. Examples of suitable therapeutic agents include, but are not limited to, antibiotics, steroids, polynucleotides (e.g., genomic DNA, cDNA, mRNA, double-stranded RNA, and antisense oligonucleotides), plasmids, peptides, peptide fragments, small molecules (e.g., doxorubicin) and other biologically active macromolecules such as, for example, proteins and enzymes.

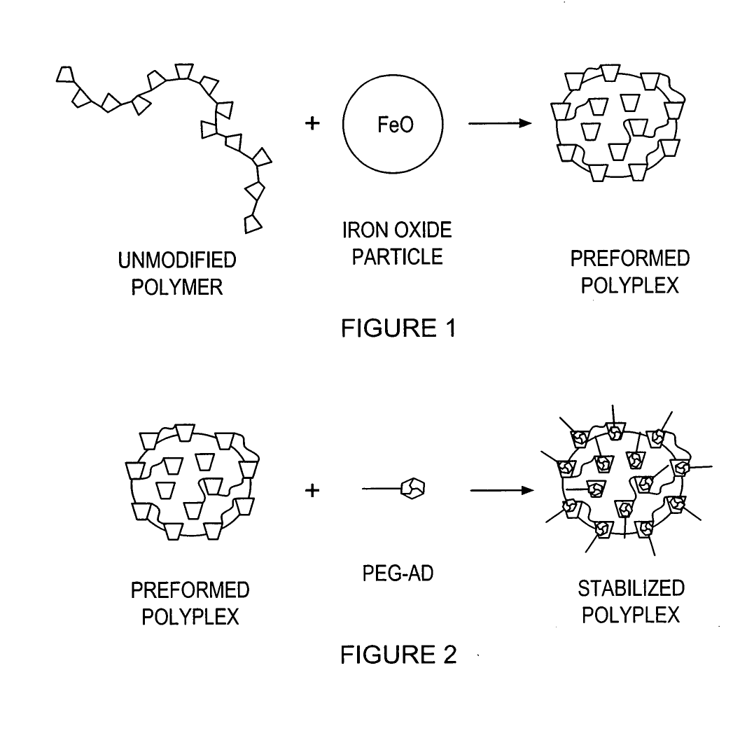

A schematic depiction of the combination of an unmodified polymer with an iron oxide paramagnetic particle is shown in FIG. 1. This process yields a preformed polyplex, in which the particle is non-covalently coated with the polymer. Prior to coating the paramagnetic particle, the unmodified polymer may first be modified by the covalent addition of one or more targeting ligands, therapeutic agents, or carrier ligands, or any combination thereof. The use of an iron oxide as the paragmentic particle is for purposes of illustration, and is not meant to limit the scope of particles envisioned by the present invention.

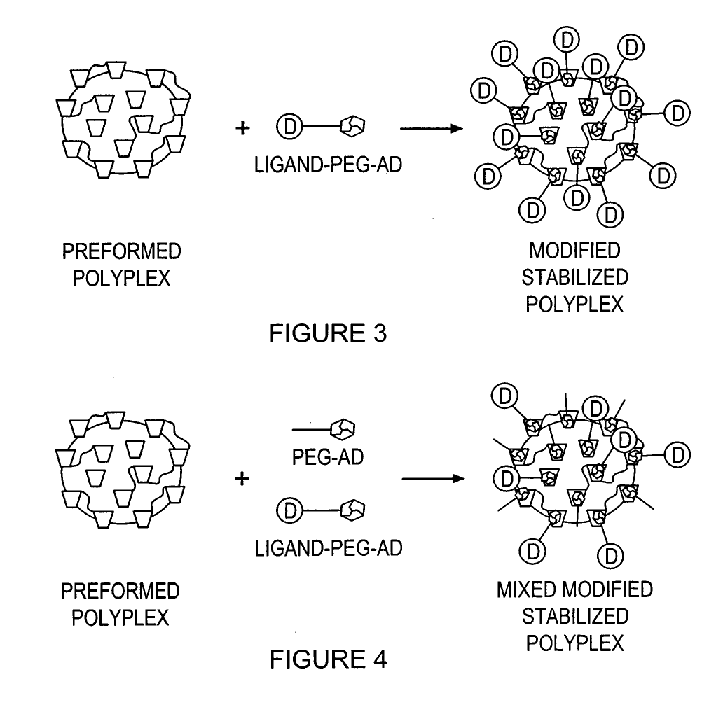

The preformed polyplex can be modified by the addition of one or more inclusion guests, each guest comprising a first portion that forms an inclusion complex with the host moieties of the polymer and a second portion that comprises a surface-modifying group (FIG. 2). Without intending to limit the scope of the invention, in this example adamantane is the inclusion guest and polyethylene glycol (PEG) is the surface-modifying group. As discussed above, the surface-modifying group may further comprise a ligand, such as a targeting ligand or a therapeutic agent (FIG. 3). The preformed polyplex may be modified with any combination of inclusion guests attached to any surface-modifying group, each optionally comprising any type of ligand (FIG. 4).

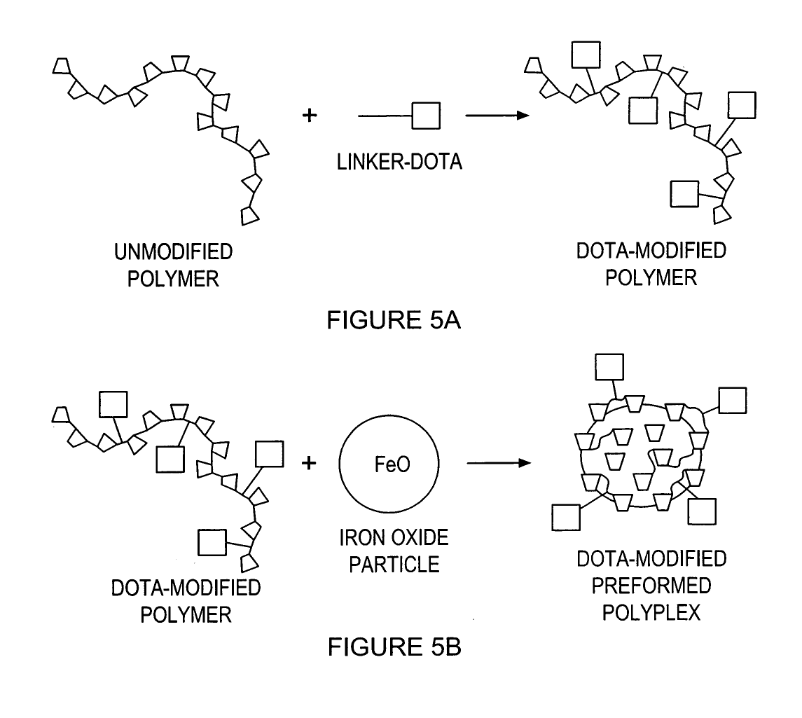

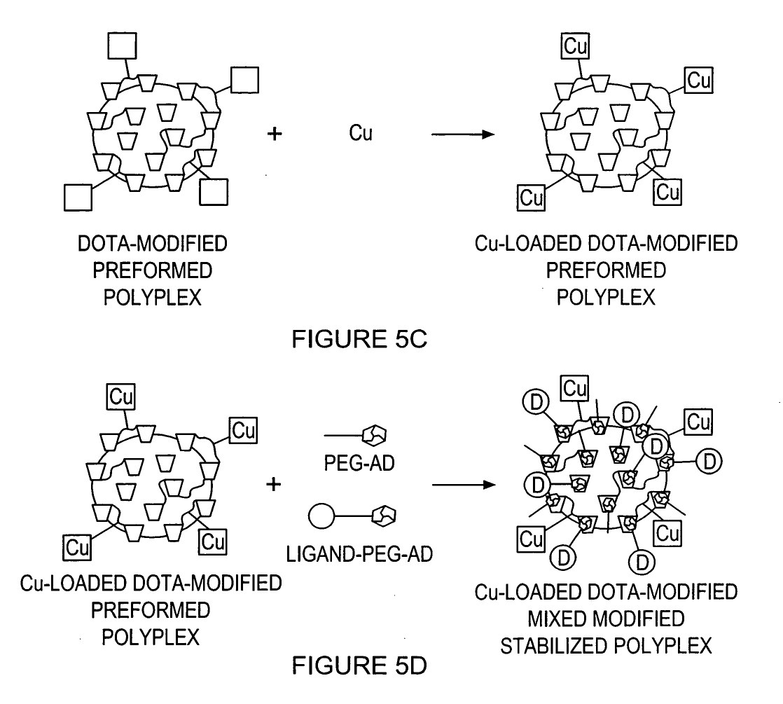

The unmodified polymer may be modified to include any type of ligand covalently attached through any type of linker (FIG. 5, Step A). In the illustration, the ligand is a carrier ligand, such as 1,4,7,10-tetraazacyclododecane-N,N′,N″,N′″-tetraacetic acid (DOTA). Once the modified polymer is formed, it can be combined with a paramagnetic particle to yield a modified preformed polyplex (FIG. 5, Step B). In the case where the ligand is a carrier ligand, the modified preformed polyplex can be combined with a radioisotope, such as copper-64, resulting in a preformed polyplex in which the radioisotope is chelated by the carrier ligand (FIG. 5, Step C). The modified preformed polyplex may be modified with any combination of inclusion guests attached to any surface-modifying group, each optionally comprising any type of ligand (FIG. 5, Step D).

(g) Selection and Administration of Contrast Agents

Contrast media can be used for resolving adjacent tissues which are similar upon imaging but histologically or physiologically different, and in functional studies of organs such as the kidney. Several classes of compounds have been explored as potential contrast agents. For MRI, these classes include superparamagnetic iron oxide particles, nitroxides, and paramagetic metal chelates. See, Mann J. S. and Brasch R. C. in HANDBOOK OF METAL-LIGAND INTERACTIONS IN BIOLOGICAL FLUIDS: BIOINORGANIC MEDICINE VOL. 2, Berthon, G., ed., Marcel Dekker, Inc., New York, N.Y. (1995). For CT scans, these classes include iodinated hydrocarbons, such as benzene rings. For positron emission tomography and radionuclide imaging, these classes include short lived radioisotopes.

Because the capillary endothelium of tumors and injured tissues exhibit high permeability rates relative to normal tissue, (MRI contrast media) MCM passively diffuses into these tissues. The poorly developed or absent lymphatic system of tumors and some tissues limits the rate of movement of macromolecules out of these tissues. This combination (enhanced permeability and retention) is used during imaging of these tissues. The tumors and injured tissues are seen by imaging as a time-dependent increased intensity in the interstitial space (Mann and Brasch, supra). The prolonged retention within the vascular compartment of tumors and some injured tissues provides nearly a constant level of enhancement for more than one hour after administration.

In MRI, contrast media improve the image obtained by altering T₁ and T₂ of hydrogen protons. In the presence of an external magnetic field, protons produce a weak fluctuating field which is capable of relaxing neighboring protons. This situation is dramatically altered in the presence of a strong paramagnet (such as a contrast agent). A single unpaired electron in a contrast agent induces a field which is nearly 700 times larger than that produced by protons and fluctuates with a frequency component which is in a range that profoundly affects both the T₁ and T₂ values of nearby protons. Thus in a T₁-weighted imaging sequence, the paramagnetic contrast media causes the protons of nearby hydrogen nuclei to release far greater amounts of energy to reach equilibrium after a r-f pulse and appear as very bright areas in an MR image. In a T₂-weighted image, the protons in tissues that take up the contrast medium release less energy to reach equilibrium and appear darker in an MRI.

Normally, paramagnetic lanthanides and transition metal ions are toxic in vivo. Therefore, it is necessary to incorporate these compounds into chelates with organic ligands. Acceptable chelates include: 1,4,7,10-tetraazacyclododecane-N,N′,N″,N′″-tetraacetic acid (DOTA); 1,4,7,10-tetraazacyclododecane-N,N′,N″-triacetic acid (DO3A), 1,4,7-tris(carboxymethyl)-10-(2-hydroxypropyl)-1,4,7,10-tetraazacyclododecane (HP-DO3A), diethylenetriaminepentaacetic acid (DPTA).

Paramagnetic metals of a wide range are suitable for chelation. Suitable metals are those having atomic numbers of 22-29 (inclusive), 42, 44 and 58-70 (inclusive), and having oxidation states of 2 or 3. Those having atomic numbers of 22-29 (inclusive), and 58-70 (inclusive) are preferred, and those having atomic numbers of 24-29 (inclusive) and 64-68 (inclusive) are more preferred. Examples of such metals are chromium (III), manganese (II), iron (II), cobalt (II), nickel (II), copper (II), praseodymium (III), neodymium (III), samarium (III), gadolinium (III), terbium (III), dysprosium (III), holmium (III), erbium (III) and ytterbium (III). Chromium (III), manganese (II), iron (III) and gadolinium (III) are particularly preferred, with gadolinium (III) the most preferred. See published PCT application WO 94/27498. Gadolinium (Gd) is a lanthanide metal with an atomic weight of 157.25 and an atomic number of 64. It has the highest thermal neutron capture cross-section of any known element and is unique for its high magnetic moment (7.98 at 298° K.). This is reflected in its seven unpaired electrons (CRC HANDBOOK OF CHEMISTRY AND PHYSICS, 75TH ED., Lide, D. R., ed., 1995).

Typically, the administration of contrast media for imaging tumors is parenteral, e.g., intravenously, intraperitoneally, subcutaneously, intradermally, or intramuscularly. Thus, the invention provides compositions for parenteral administration which comprise a solution of contrast media dissolved or suspended in an acceptable carrier, preferably an aqueous carrier. The concentration of MCM varies depending on the strength of the contrast agent but typically varies from 0.1 μmol/kg to 100 μmol/kg. A variety of aqueous carriers may be used, e.g., water, buffered water, 0.9% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

Exemplification

Preparation of Uncrosslinked Anionic Cyclodextrin Polymer

Cross-linked anionic polymer (1.5 g) is dissolved in methanol (10 mL), followed by the addition of aqueous NaOH (1M, 10 mL). The reaction is stirred for 15 h at room temperature. The methanol is removed by vacuum. The remaining solution is frozen, and the water is removed by lyophilization. The solution is resuspended in water (10 mL) and dialyzed against water using a 7K MWCO dialysis cartridge (Pierce). The polymer solution is lyophilized to remove the water, resulting in a white, fluffy powder.

Preparation of Cyclodextrin-Polymer Coated Iron Oxide Paramagnetic Particles

Iron oxide particles, encapsulated in a linear, anionic cyclodextrin polymer, are prepared by an aqueous phase coprecipitation method, similar to a process described in U.S. Pat. No. 5,262,176 for the preparation of iron oxide nanoparticles coated with dextran. To prepare larger 90 nm particles, cross-linked, anionic cyclodextrin polymer (100 kDa) (5.125% w/v) is dissolved in 5 mL of degassed double distilled H₂O. Alternatively, to make 30 nm particles, uncrosslinked anionic cyclodextrin polymer* (80 kDa) (6% w/v) is dissolved in degassed water. Iron III chloride hexahydrate (0.12 M) is added to the cyclodextrin polymer mixture and magnetically stirred under argon. Next, 0.063 g of iron (II) chloride heptahydrate is dissolved in 215 μL of degassed ddH₂0. The iron II chloride solution is added to the mixture containing cyclodextrin polymer and iron (III) chloride such that the final reaction mixture contains a 2:1 molar ratio of Fe³⁺ to Fe²⁺. The solution is cooled to 0-4° C. under argon. An aqueous solution of 28% ammonium hydroxide (225 μL) is added dropwise to the reaction. The solution is heated slowly to 80° C. over 45 minutes, and the temperature is maintained at 80° C. for 75 minutes with stirring.

The solution is cooled to room temperature by removing the heat source. After cooling, the solution is spun in a centrifuge at 3200 rpm for 10 min. Precipitated material is discarded, and the supernatant, which contains CD polymer coated iron oxide particles, is mixed with ammonium citrate buffer pH 8.2 (1 mM and 10 mM for larger particles and smaller particles, respectively) in a 1:1 ratio of buffer to supernatant. To remove excess cyclodextrin polymer and ammonium hydroxide base, the solution is purified by ultrafiltration in an Amicon Ultra 4 MWCO 100K unit and centrifuged at 3200 rpm for 15 min. The concentrate is mixed with an equal volume of ammonium citrate buffer, and the ultrafiltration step is repeated twice. The resulting solution contains iron oxide nanparticles coated with cyclodextrin polymer in citrate buffer at pH 8.2.

The concentration of iron in the particles can be determined by measuring absorbance at 356 nm. The validity of this technique was confirmed by measuring the iron content of the particles by ICP-MS. The concentration of polymer in the final solution is determined by a phenol-sulfuric acid assay. The composition of the final nanoparticle solution is typically 20-25 mg/mL of polymer per 1 mg/mL of iron for the larger F3 particles, and the composition of the final nanoparticle solution is typically 5-10 mg/mL of polymer per 1 mg/mL of iron for the smaller F1 particles.

Particle Modification

After the cyclodextrin-polymer coated nanoparticles are prepared, the outer surface the nanoparticles can be modified by mixing the nanoparticles with adamantane-PEG (AD-PEG) at a 1:1 molar ratio of cyclodextrin to AD-PEG. Adamantane interacts with the polymer by forming a stable inclusion complex with cyclodextrin, and PEG is exposed to the solvent, which stabilizes the nanoparticles under physiological conditions.

The outer surface of the nanoparticles can be further modified by the attachment of a ligand to adamantane-PEG. For example, transferrin targeting ligand is mixed with the nanoparticle solution at 1.7% w/w of AD-PEG-Tf to AD-PEG. Transferrin (Tf) is covalently attached to AD-PEG. When AD-PEG-Tf is mixed with the particles, the transferrin targeting ligand is displayed on the outside of the nanoparticle. In their final form, the nanoparticles have an iron oxide core ensheathed inside of the cyclodextrin polymer, PEG, and a targeting ligand displayed on the outside of the complex. This methodology allows for the preparation of superparamagnetic iron oxide MRI agents with sizes and surface properties almost identical to therapeutic delivery particles.

Preparation of Dual MRI-PET Imaging Probles

A PET probe, ⁶⁴Cu or ⁶⁷Cu, can be attached easily to the CD-coated MRI contrast agents. This enables in vivo localization of the probe to be monitored by both MRI and PET. In the first step, a chelator molecule is covalently coupled to the CD polymer backbone. In the present example, DOTA (1,4,7,10-tetraazacyclododecane-1,4,7,10-tetraacetic acid) was attached to the cyclodextrin polymer by linking a carboxyl group of the DOTA molecule to a carboxyl group of the polymer backbone through a hexaethyldiamine linker molecule. In the presence of EDC(N-3-dimethyl-aminopropyl-N′-ethycarbodiimide) and sulfo-NHS(N-Hydroxysulfosuccinimide), an amide bond forms between the carboxyl groups of DOTA and CD-polymer and the amine moieties of the hexaethyldiamine linker, resulting in a stable linkage.

The incorporation of DOTA onto the polymer can be quantified by measuring the spectrophotometric shift of the compound in the presence of Cu²⁺. When Cu²⁺ binds to DOTA, the resulting complex has an absorbance peak at 730 nm, which is not present for either compound individually.

The DOTA-conjugated CD-polymer was used to prepare iron oxide nanoparticles using the same method described above for preparing polymer-coated particles. The resulting iron oxide nanoparticles were coated in DOTA-cyclodextrin polymer. The particles were measured to be 30 nm in diameter by photo correlation spectroscopy (PCS), indicating that DOTA does not interfere with iron oxide nanoparticle formation or coating.

Dynamic Light Scattering (DLS) and Zeta Potential

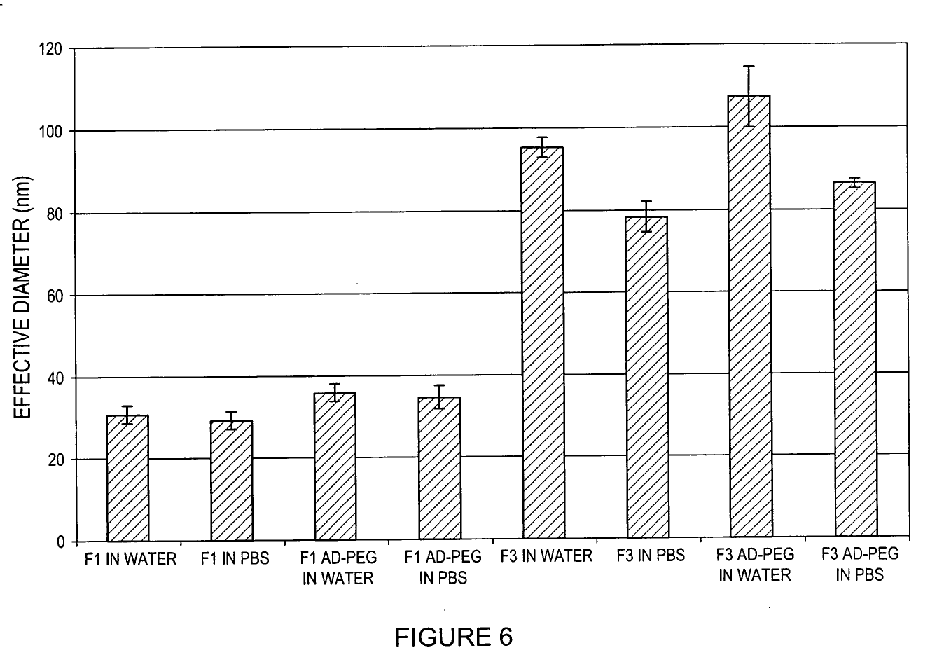

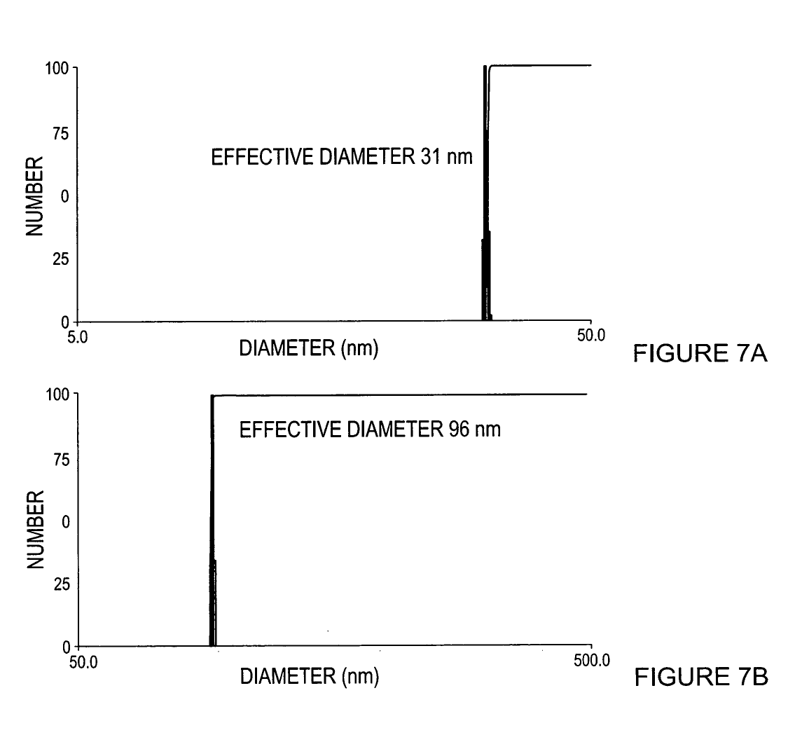

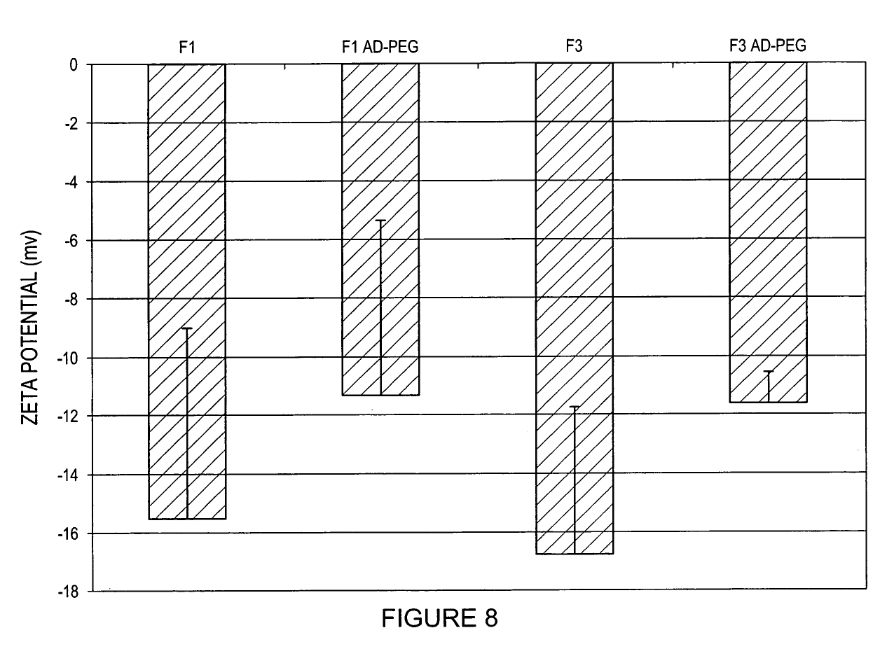

The hydrodynamic diameter of the particles was measured by dynamic light scattering, using a ZetaPALS dynamic light scattering detector (DLS, Brookhaven Instruments Corporation). One population of particles has a hydrodynamic diameter of 30 nm, and the other population has a hydrodynamic diameter of 90 nm as shown in FIGS. 6 and 7. The zeta potential of the particles was calculated from the electrophoretic mobilities using the Smoluckowski equation. For both sizes of particles, the zeta potential of the unpegylated particles is −16 mv, and the zeta potential of the pegylated particles is −11 mv (FIG. 8).

Transmission Electron Microscopy (TEM)

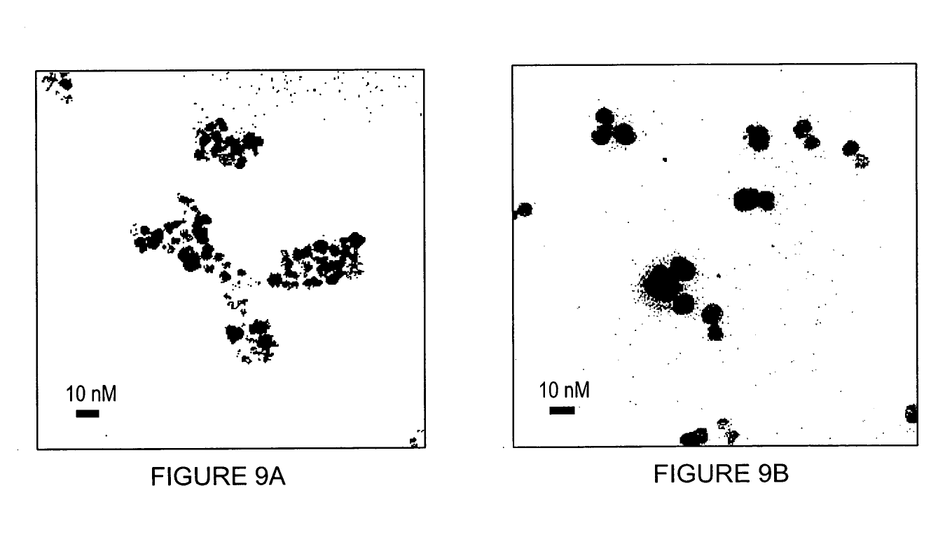

The diameter of the iron oxide core was measured by TEM. An aqueous drop of nanoparticle solution was placed on a carbon coated copper grid, and the excess liquid was wicked away. The nanoparticles visualized under an 80 kV electron beam. The iron oxide cores are visible in the FIG. 9, while the polymer coating is fully transparent to the electron beam and therefore invisible on the images. The larger particles (90 nm in diameter by DLS) have a 10 nm diameter iron oxide core, while the smaller particles (30 nm in diameter by DLS) have a 5 nm diameter iron oxide core.

Photon Correlation Spectroscopy (PCS)

The hydrodynamic diameter of the particles was measured by photon correlation spectroscopy. One population of particles (F1) has a hydrodynamic diameter of 30 nm, and the other population (F3) has a hydrodynamic diameter of 90 nm.

Magnetic Properties

Monocrystalline (3-20 nm diameter) iron oxide nanoparticles are superparamagnetic, which means that they become magnetized in an external magnetic field. The effect of the nanoparticles on the local magnetic environment was evaluated by imaging the particles in a 9.4T horizontal bore magnet. T₂ or the spin-spin relaxation time was measured at varying concentrations of the particles in water. The concentration dependence on T₂ relaxation times is quantified by plotting the particle concentration versus 1/T₂. The slope of the line, or the relaxivity (R2), is a measure of proton relaxation enhancement. Higher relaxivity values indicate that the contrast agent has a strong concentration-dependent effect on decreasing the T₂ relaxation times and is a powerful contrast agent.

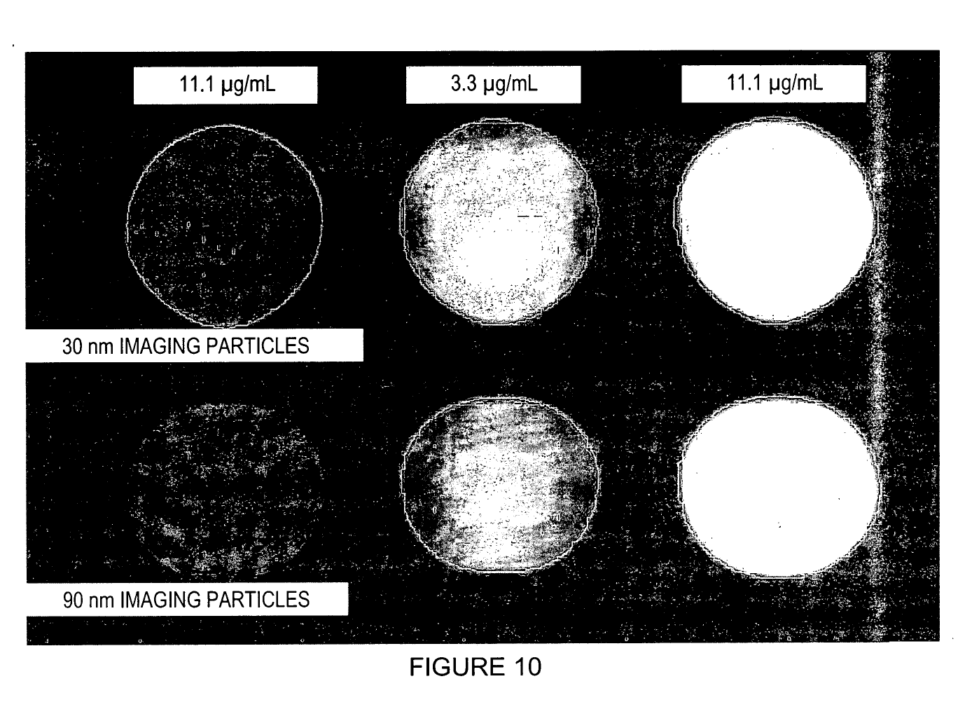

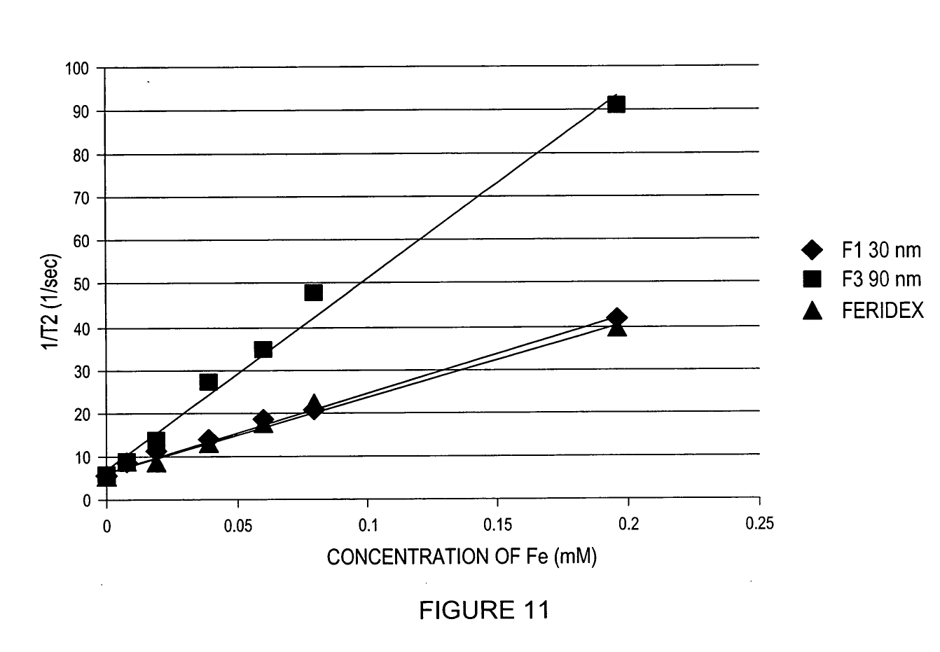

The larger particles (90 nm) have a more pronounced T₂ effect than smaller particles (FIG. 10). The relaxivity of the 30 nm contrast agent is 179 mM⁻¹s⁻¹, while the relaxivity of the 90 nm contrast agent is 439 mM⁻¹s⁻¹. (FIG. 11). This is not surprising given that the T₂ relaxation effect is proportional to the diameter of the iron oxide core. The greater relaxivity observed for the 90 nm nanoparticle compared to the 30 nm nanoparticles is consistent with the size of the iron oxide core observed by TEM.

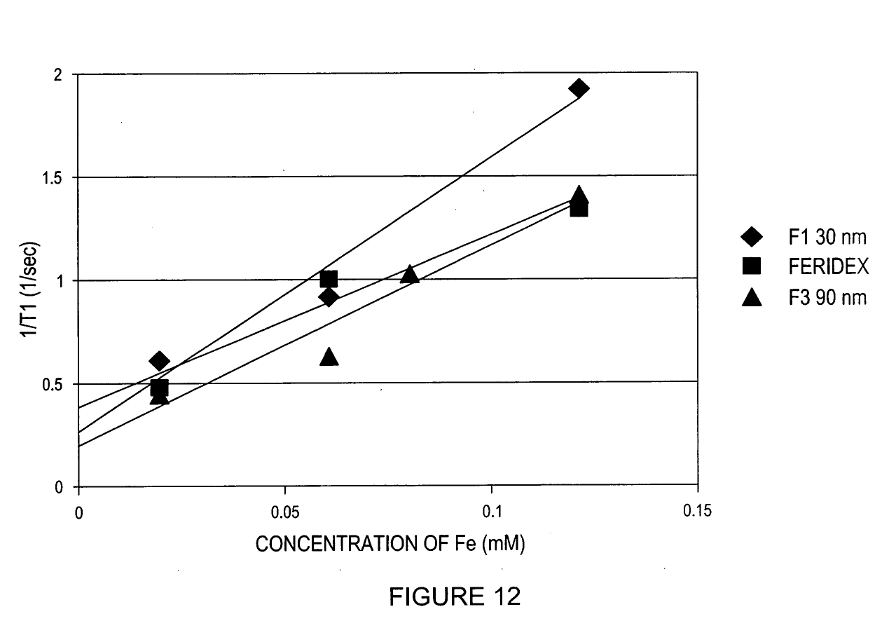

We compared the magnetic properties of our particles to a commercially available contrast agent, Feridex. Feridex is composed of a 5 nm diameter iron oxide core that is coated with dextran; it has a hydrodynamic diameter of 30-40 nm. The relaxivity value of Feridex was measured as 176 mM⁻¹s⁻¹, which is nearly identical to the T₂ relaxivity of our 30 nm contrast agent (179 mM⁻¹s⁻¹) (FIG. 12).

The concentration dependence on T₁ relaxation is quantified by plotting the particle concentration versus 1/T₁. The slope of the line, or the relaxivity (R1), is a measure of proton relaxation enhancement. Higher relaxivity values indicate that the contrast agent has a strong concentration-dependent effect on decreasing the T₁ relaxation times.

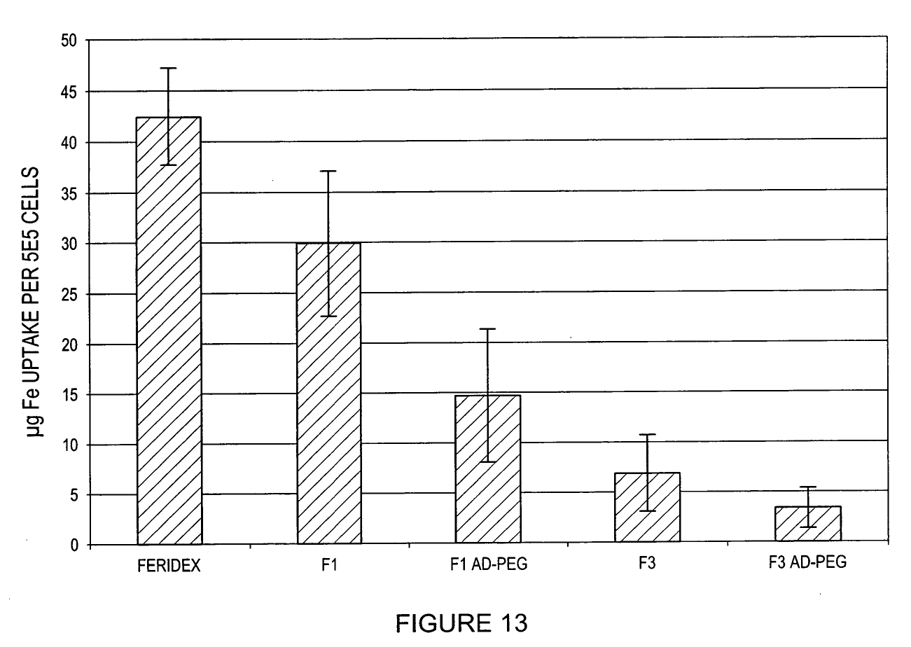

The particles were found to enhance T₁ (spin-spin) relaxation times, but to a lesser extent than the T₂ relaxation enhancement effect. The relaxivity (R1) of our 30 nm particles, our 90 nm particles, and Feridex are 13.2 mM⁻¹s⁻¹, 9.8 mM⁻¹s⁻¹, 8.4 mM⁻¹s⁻¹, respectively (FIG. 13). T₁ relaxation time does not depend on the cross-sectional area of the nanoparticles, so it is not surprising that the T₁ relaxation times are similar for all three contrast agents tested.

In Vitro Macrophage Uptake

RAW264.7 (mouse macrophage) cells were grown in DMEM supplemented with 10% fetal bovine serum and 1× antibiotic/antimycotic solution. Cells (5×10⁵/mL) were suspended in DMEM and seeded onto a 24-well plate with 1 mL of cell suspension added to each well. Nanoparticles were added to each well to a final concentration of 0.1 mg Fe/mL. After 15 h, the media was removed, and the cells were washed three times with Hank's Balanced Salt Solution. The cells were lysed by addition of cell lysis buffer (Promega). Iron uptake was determined by measuring the absorbance at 356 nm of the lysed cell solution (FIG. 13). F1 particles exhibit four-fold greater macrophage uptake than F3 particles. Pegylated particles exhibit half the macrophage uptake as unpegylated particles. Particle uptake in Neuro2A cells was not detected by Prussian Blue staining or by measuring absorbance at 356 nm.

Tumor Histology

Neuro2A (mouse neuroblastoma) cells or Hep3B (mouse hepatoma cells) were grown DMEM supplemented with 10% fetal bovine serum and 1× antibiotic/antimycotic solution at 37° C. and a 5% CO₂ atmosphere. Cells were washed once with PBS and suspended to 5×10⁶ cells/mL in RPMI. The cell suspension (200 μL, 10⁶ cells) was injected subcutaneously over the shoulder blade of an A/J mouse. After 13 days, A/J mice bearing a subcutaneous Neuro2A tumor on the shoulder were injected systemically with nanoparticles. Each injection was composed of 15 mg Fe/kg tissue in 5% glucose solution. Three types of nanoparticles were injected: F3, F3-AD-PEG, and F3-AD-PEG-Tf (1.7 w %). F3 particles are 90-100 nm in diameter. After 1 h post-injection, the mice were culled by CO₂, and the tumor was removed from the animal and preserved in 10% formalin. Iron was detected in the tumors by staining sections with DAB-enhanced Prussian blue stain.

In Vivo MR Imaging

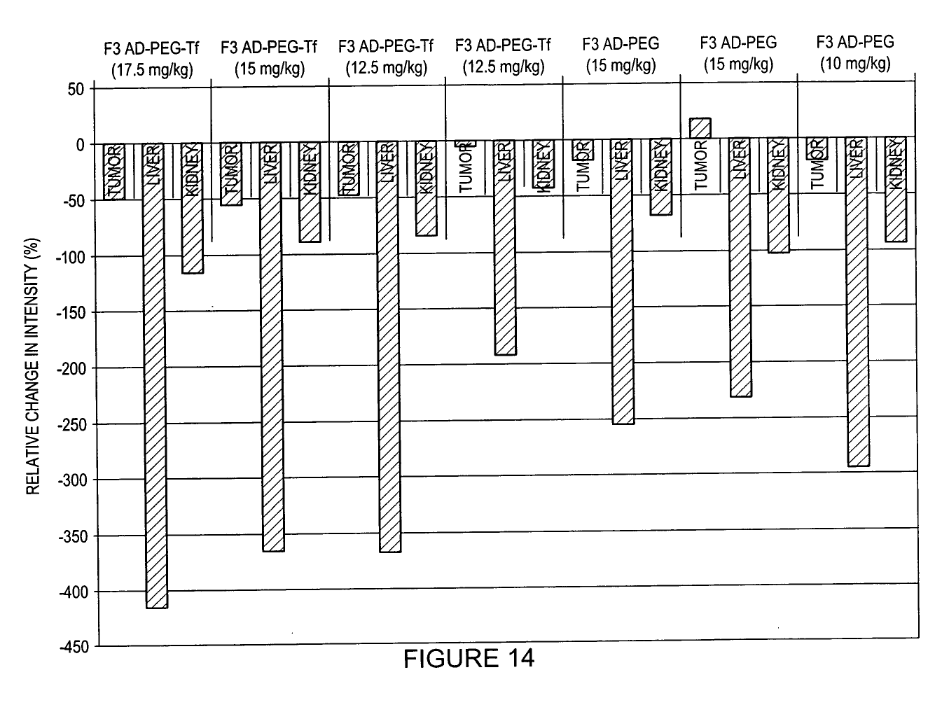

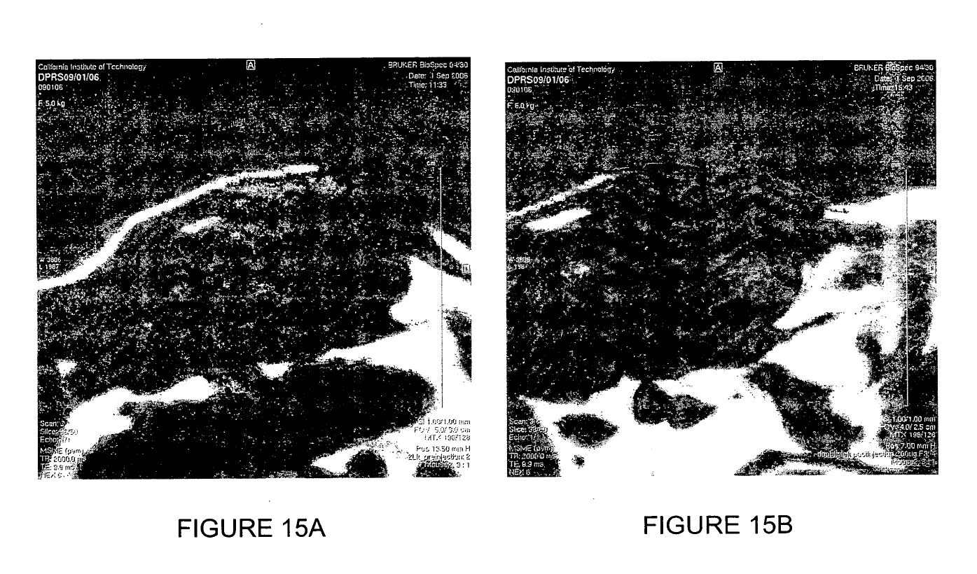

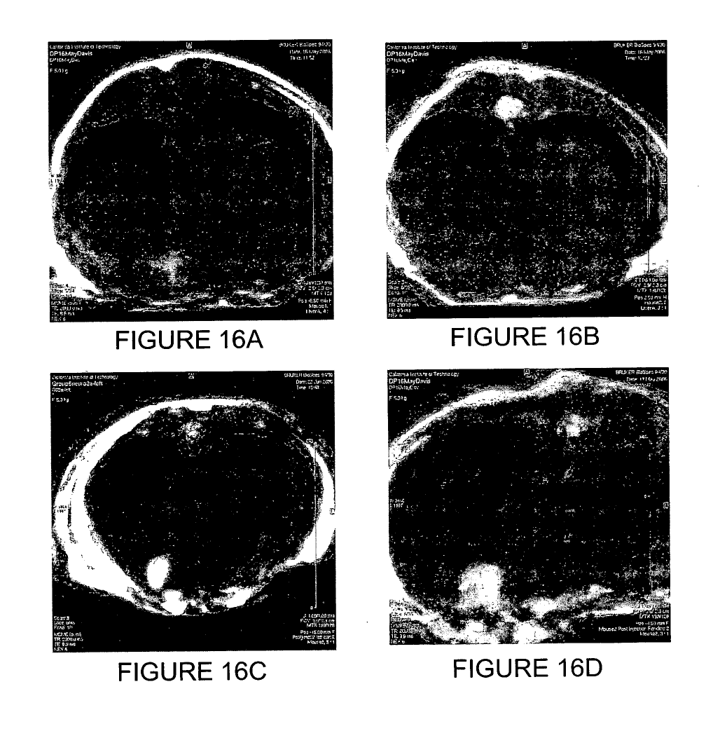

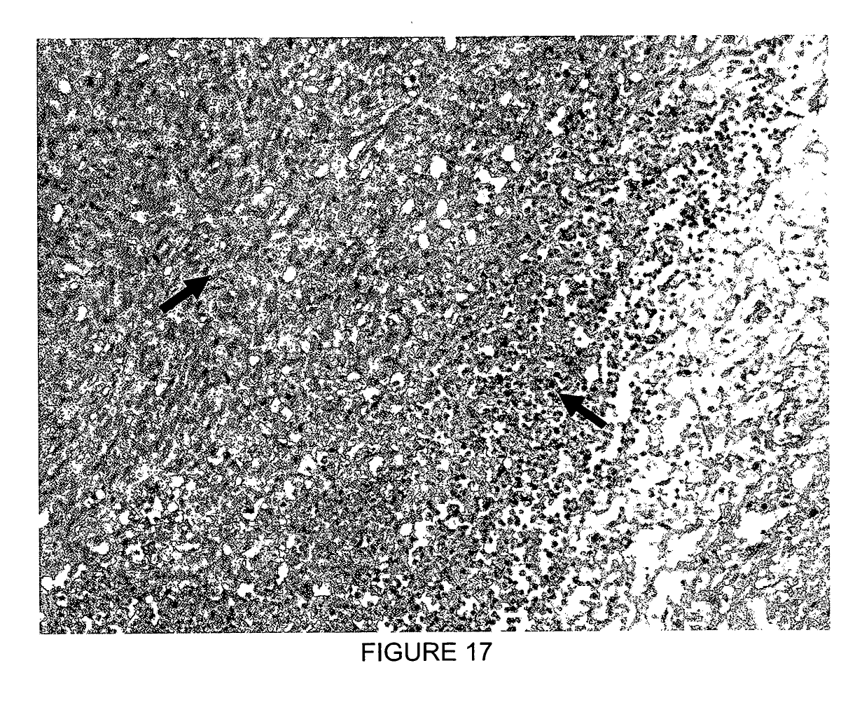

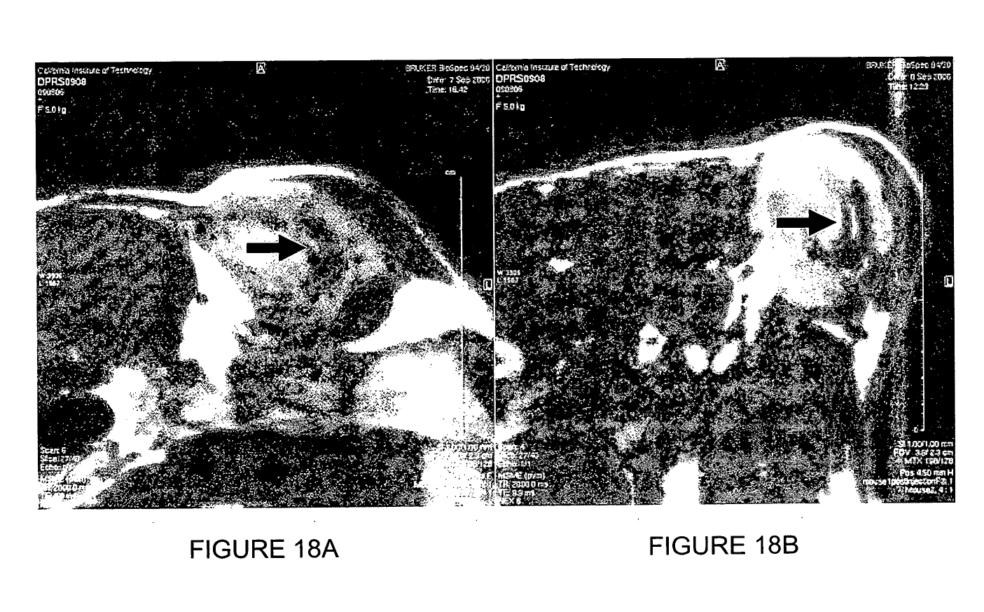

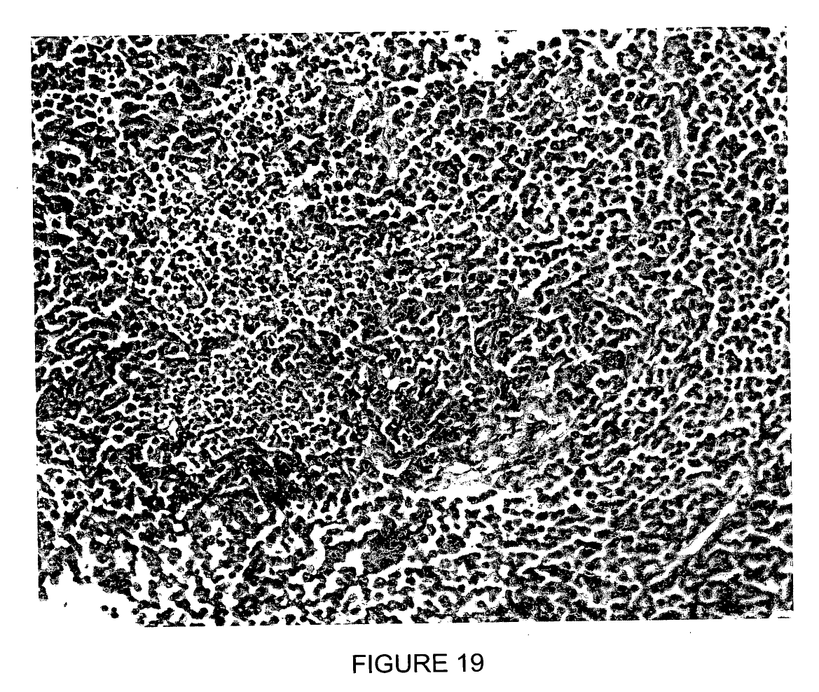

The particles were injected systemically into Neuro2A-bearing A/J mice or Hep3B-bearing scid mice. MR imaging of the mice was performed in a 9.4T magnet with a T₂-weighted spin echo sequence. The mice were scanned prior to injection of contrast agent and again one to four hours after contrast agent injection (FIGS. 14-19).

The relative signal intensity enhancement was calculated as a percentage of the drop in tissue intensity post-contrast agent enhancement relative to the signal intensity of the tissue precontrast. Image intensity was normalized between pre- and post-contrast images by determining the intensity of the desired tissue relative to muscle tissue. The built-in Paravision Imaging processing Tool software was used to measure signal intensities of chosen ROIs in the desired tissue: liver parenchyma, tumor, or back muscle (FIG. 14). Signal-to-noise ratio (SNR) was calculated, and the relative drop in signal intensity (SI) was obtained using the following formula: (SI)=100×((SNR_{precontrast}−SNR_{postcontrast}) SNR_{precontrast}).

Preparation of Pet Probe or Dual MRI-Pet Imaging Probes

A PET probe, ⁶⁴Cu, is attached to the CD-coated MRI contrast agents by conjugating a metal-chelating small molecule to the polymer. DOTA (1,4,7,10-tetraazacyclododecane-1,4,7,10-tetraacetic acid) was attached to the cyclodextrin polymer by linking a carboxyl group of the DOTA molecule to a carboxyl group of the polymer backbone though a hexaethyldiamine linker molecule. In the presence of EDC(N-(3-Dimethylaminopropyl)-N′-ethylcarbodiimide) and Sulfo-NHS(N-Hydroxysulfosuccinimide), amide bonds link DOTA to the CD-polymer by using hexaethyldiamine as a linker. Briefly, cyclodextrin polymer (4 mg/mL) is suspended in water. DOTA is added to the reaction at a 16% molar ratio to carboxyl groups of the polymer backbone. Hexaethyldiamine, EDC, and Sulfo-NHS are added to the reaction at a 1:1 molar ratio to DOTA. The solution is stirred for 2 h at room temperature. To terminate the reaction and remove excess DOTA, hexaethyldiamine, EDC, and sulfo-NHS, the solution is purified by ultrafiltration in an Amicon Ultra 15 MWCO 10K unit. The ultrafiltration unit is spun in a centrifuge at 3200 rpm until the solution is concentrated to roughly 1/10 of its initial volume.

The DOTA-conjugated CD-polymer was used to prepare iron oxide nanoparticles using the same method described above for preparing polymer-coated particles. The resulting iron oxide nanoparticles were coated in DOTA-cyclodextrin polymer. The particles were measured to be 22 nm in diameter by dynamic light scattering (DLS) indicating that DOTA does not interfere with iron oxide nanoparticle formation or coating.

The amount of DOTA incorporation onto the polymer backbone is determined by measuring the absorbance at 660 nm of the polymer in the presence of Arsenazo III and GdCl₃. This assay enables detection of free Gd (III) in solution. The concentration of free Gd is equivalent to the concentration of DOTA.

In Vivo PET Imaging

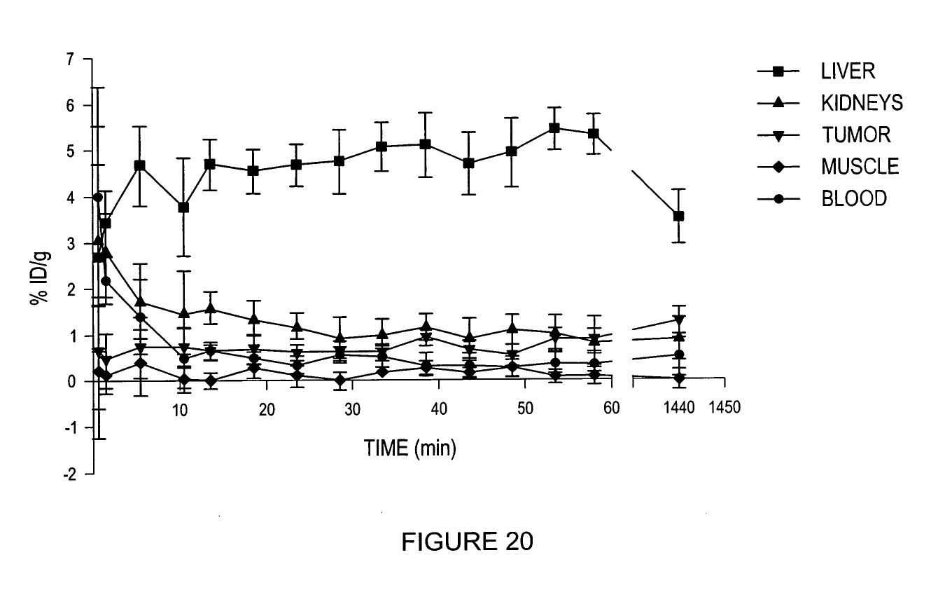

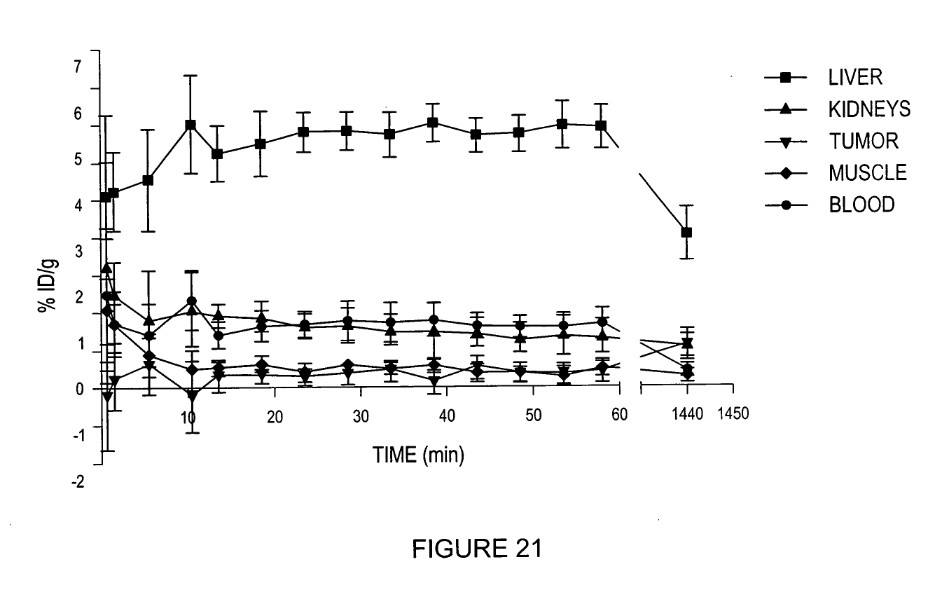

DOTA-labeled nanoparticles were mixed with ⁶⁴Cu at a 200-fold molar excess of DOTA to Cu in acetate buffer (250 mM, pH 5). The labeling reaction was carried out for 1 h at 60° C. To remove excess ⁶⁴Cu, the nanoparticles were purified by ultracentrifugation. The labeling reaction was loaded onto a Centricon YM-3 filter column (3 kDa MWCO, Millipore) and the columns were centrifuged at 10 K rpm for 15 min. The supernatant was collected and mixed with an equal volume of 10% glucose. Because the dose of imaging particles needed for PET (0.01 mg Fe/kg tissue) is much lower than the dose of particles needed for MRI (˜10 mg Fe/kg tissue), unlabeled nanoparticles were sometimes mixed into the injected dose (FIGS. 20-21)

### REFERENCES

Additional Cyclodextrin-Containing Polymers that can be Modified According to the teachings of the present invention, as well as methods of preparing such polymers and compositions comprising such polymers, are disclosed in U.S. Pat. Nos. 4,770,183, 5,262,176, 6,509,323, 6,884,789, 7,018,609, 7,091,192, and 7,166,302, U.S. application Ser. Nos. 10/372,723, 10/656,838, 11/358,976, 11/321,441, and PCT Application No. WO00/33885 and WO02/49676, all of which are hereby incorporated herein by reference in their entireties.

All of the references, patents, and publications cited herein are hereby incorporated by reference.

### EQUIVALENTS

Those skilled in the art will recognize, or be able to ascertain using no more than routine experimentation, numerous equivalents to the compounds and methods of use thereof described herein. Such equivalents are considered to be within the scope of this invention and are covered by the following claims.

1. A paramagnetic particle coated by a cyclodextrin-containing polymer.

2. The paramagnetic particle of claim 1, wherein the cyclodextrin-containing polymer has a linear polymer backbone, comprising a plurality of substituted or unsubstituted cyclodextrin moieties and linker moieties, wherein each of the cyclodextrin moieties is attached to up to two of said linker moieties, each linker moiety covalently linking up to two cyclodextrin moieties.

3. The paramagnetic particle of claim 2, wherein the cyclodextrin polymer comprises n′ units of U, wherein n′ represents an integer in the range of 1 to about 30,000; and U is represented by the general formula: 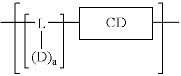
wherein,
CD represents a cyclodextrin molecule, or derivative thereof;
L represents a linker group;
D, independently for each occurrence, represents a targeting ligand, a therapeutic agent or prodrug thereof, or a carrier ligand;
a, independently for each occurrence, represents an integer in the range of 0 and 10.

4. The paramagnetic particle of claim 1, wherein a surface of the polymer is modified by one or more inclusion guests, each inclusion guest comprising a first portion that forms an inclusion complex with the cyclodextrin moieties of the polymer, and a second portion that comprises a surface-modifying group.

5. The paramagnetic particle of any of claims 1-4, wherein the cyclodextrin-containing polymer coats a plurality of paramagnetic particles.

6. The paramagnetic particle of any of claims 1-5, wherein the paramagnetic particle comprises a metal oxide or metal mixed oxide.

7. The paramagnetic particle of claim 6, wherein the metal is iron.

8. The paramagnetic particle of any of claims 1-5, wherein the particle has an average hydrodynamic diameter in the range of 10-100 nanometers.

9. The paramagnetic particle of claim 3, wherein a plurality of occurrences of D independently represent a targeting ligand.

10. The paramagnetic particle of claim 3, wherein a plurality of occurrences of D independently represent a therapeutic agent.

11. The paramagnetic particle of claim 3, wherein D is attached to the cyclodextrin polymer by a linker that comprises a biohydrolyzable bond.

12. The paramagnetic particle of claim 11, wherein the biohydrolyzable bond is selected from an ester, amide, carbonate, or a carbamate.

13. The paramagnetic particle of claim 3, wherein D, independently for each occurrence, is a carrier ligand.

14. The paramagnetic particle of claim 13, wherein the carrier ligand is a radioisotope carrier.

15. The paramagnetic particle of claim 4, wherein the inclusion guest is adamantane.

16. The paramagnetic particle of claim 4, wherein the inclusion guest is covalently attached to a surface-modifying group.

17. The paramagnetic particle of claim 16, wherein the surface-modifying group is a stabilizing group.

18. The paramagnetic particle of claim 16, wherein the stabilizing group is selected from phosphate, diphosphate, carboxylate, polyphosphate, thiophosphate, phosphonate, thiophosphonate, sulphate, sulphonate, mercapto, silanetriol, trialkoxysilane-containing polyalkylene glycols, carbohydrate or phosphate-containing nucleotides, oligomers thereof or polymers thereof.

19. The paramagnetic particle of claim 16, wherein the surface-modifying group further comprises a ligand.

20. The paramagnetic particle of claim 17, wherein the ligand is a targeting ligand or a therapeutic agent or prodrug thereof.
